(19) United States
(12) Patent Application Publication
(10) Pub. No.: US 2003/0217378 A1
(43) Pub. Date: Nov. 20, 2003

(54) Cloning using rapidly matured oocytes

(75) Inventors: Steven L. Stice, Athens, GA (US); Kazuchika Miyoshi, Kagoshima (JP)

Correspondence Address: KING & SPALDING, 191 PEACHTREE STREET, N.E., ATLANTA, GA, 30303-1763, US

(73) Assignee: The University of Georgia Research Foundation, Inc, Atlanta, GA

(21) Appl. No.: 10/406,483

(22) Filed: Apr. 2, 2003

Related U.S. Application Data
(60) Provisional application No. 60/370,586, filed on Apr. 5, 2002.

(51) Int. Cl. A01K 67/027

(52) U.S. Cl. 800/15; 800/21; 800/17

(57) ABSTRACT

A method of producing a cloned or genetically modified non-human mammalian embryo comprising: (a) providing a cell culture comprising a plurality of in vitro matured oocytes; (b) preferentially selecting from the cell culture a rapidly matured oocyte or developmentally competent oocyte; (c) transferring DNA from a donor cell derived from non-human mammalian tissue to the matured oocyte to form a nuclear transfer unit; and (d) culturing said nuclear transfer unit to form an embryo. At the initiation of maturation, oocytes are preferably beyond the GV-II stage of prophase I. Porcine oocytes most preferably mature in about 20-28 hours.

### FIELD OF THE INVENTION

This invention is in the field of nuclear transfer technologies, and the use of nuclear transfer in embryo and animal cloning. In particular, this invention relates to the use of oocytes with improved developmental competence in the nuclear transfer process, and to the generation of cloned embryos and animals including genetically selected and/or modified animals from oocytes with improved developmental competence.

### BACKGROUND OF THE INVENTION

Nuclear transfer is the process of removing genetic material from an unfertilized oocyte (typically haploid material from a MII oocyte), inserting genetic material from a donor cell into the oocyte (typically diploid material from a cell in G1 or G1/G0), and activating the oocyte to initiate cell division and growth. Several permutations to this general theme exist. For example, the donor cell can be an embryonic cell, a somatic cell, or an adult cell, and can derive from various animal tissues. Also, it is not absolutely necessary to remove the haploid genetic material from the oocyte before inserting the donor genetic material. Similarly, there are various ways to activate renucleated oocytes, and in some circumstances oocytes can spontaneously initiate cell division and growth even without artificial activation. Nevertheless, the general principles of inserting a complete set of genetic material into an oocyte, and growing an embryo, fetus, and live animal that incorporate the inserted genetic material, remain the same.

The promise of nuclear transfer resides in its use to propagate cells, tissues, and entire animal beings that have the same genetic makeup as a cell, tissue, or animal already in existence. The technology thus facilitates several burgeoning industries, including:

Cloning of animals with superior genetic makeup for use as breeding stock in livestock production operations.

Production of genetically modified transgenic animals for various industrial and medical uses, including transgenic protein production and xenographic organ, tissue, and cell transplantation.

Production of stem cells that are identical in genetic makeup to the genetic makeup of a diseased individual, for use in various medical treatments of the diseased individual.

The cloning of various animals created by transferring differentiated cells into enucleated oocytes has been reported in the literature (Sheep—Campbell, et al., Sheep cloned by nuclear transfer from a cultured cell line, Nature 1996, 380: 64-66; Wilmut, et al., Viable offspring derived from fetal and adult mammalian cells, Nature 1997, 385: 810-813; Cattle—Cibelli, et al., Cloned transgenic calves produced from nonquiescent fetal fibroblasts, Science 1998, 280: 1256-1258; Kato, et al., Eight calves cloned from somatic cells of a single adult, Science 1998, 282: 2095-2098; Wells, et al., Production of cloned calves following nuclear transfer with cultured adult mural granulosa cells, Biol Reprod 1999, 60: 996-1005; Mouse—Wakayama, et al., Full-term development of mice from enucleated oocytes injected with cumulus cell nuclei, Nature 1998, 394: 369-374; Goat—Baguisi, et al., Production of goats by somatic cell nuclear transfer, Nature Biotech 1999, 17: 456-461; Pig—Onishi, et al., Pig cloning by microinjection of fetal fibroblast nuclei, Science 2000, 289: 1188-1190; Polejaeva, et al., Cloned pigs produced by nuclear transfer from adult somatic cells, Nature 2000, 407: 86-90; Betthauser, et al., Production of cloned pigs from in vitro systems, Nature Biotech 2000, 18: 1055-1059). In these studies, both in vivo- and in vitro-matured oocytes have been used as recipients. The in vitro oocyte maturation system is typically employed because immature oocytes can be obtained from slaughtered animals, which provide an abundant and stable supply of recipient oocytes. In vitro-matured oocytes have been commonly used for production of cloned calves, Cibelli, et al. (1998) (supra); Kato, et al. (1998) (supra); Wells, et al. (1999) (supra). Similarly, several studies have reported successful development of porcine embryos reconstituted with in vitro-matured oocytes to the blastocyst stage, Betthauser, et al. (2000) (supra); Tao, et al., Development of pig embryos by nuclear transfer of cultured fibroblast cells, Cloning 1999, 1: 55-62; Tao, et al., Development of pig embryos reconstructed by microinjection of cultured fetal fibroblast cells into in vitro matured oocytes, Anim Reprod Sci 1999, 56: 133-141; Miyoshi, et al., Establishment of a porcine cell line from in vitro-produced blastocysts and transfer of the cells into enucleated oocytes, Biol Reprod 2000, 62: 1640-1646; Miyoshi, et al., Improvement in development of porcine embryos reconstituted with cells from blastocyst-derived cell lines and enucleated oocytes by optimization of reconstruction methods, Cloning 2000, 2: 175-184; Koo, et al., In vitro development of reconstructed porcine oocytes after somatic cell nuclear transfer, Biol Reprod 2000, 63: 986-992; Kuhholzer, et al., Production of transgenic porcine blastocysts by nuclear transfer, Mol Reprod Dev 2000, 56: 145-148; Uhm, et al., Expression of enhanced green fluorescent protein (EGFP) and neomycin resistant (Neo^{R}) genes in porcine embryos following nuclear transfer with porcine fetal fibroblasts transfected by retrovirus vector, Mol Reprod Dev 2000, 57: 331-337; Uhm, et al., In vitro development of porcine enucleated oocytes reconstructed by the transfer of porcine fetal fibroblasts and cumulus cells, Theriogenology 2000, 54: 559-570; Koo, et al., Developmental potential and transgene expression of porcine nuclear transfer embryos using somatic cells, Mol Reprod Dev 2001, 58: 15-21. There is only one report of cloned pigs resulting from nuclear transfer embryos using in vitro-matured oocytes, Betthauser, et al. (2000) (supra), and it has been suggested that in vivo-derived materials enhance the probability of obtaining porcine offspring, Onishi, et al. (2000) (supra); Polejaeva, et al. (2000) (supra).

Although porcine blastocysts can be produced from immature oocytes by using in vitro maturation/fertilization/culture systems, it is difficult to obtain blastocysts efficiently because of high rates of polyspermic fertilization and low developmental competence of the zygotes produced in vitro, Niwa K, Effectiveness of in vitro maturation and in vitro fertilization techniques in pigs, J Reprod Fertil Suppl 1993, 48: 49-59. Continuous advances have led to improvements in porcine blastocyst production in vitro, Abeydeera, et al., Fertilization and subsequent development in vitro of pig oocytes inseminated in a modified tris-buffered medium with frozen-thawed ejaculated spermatozoa, Biol Reprod 1997, 57: 729-734; Abeydeera, et al., Development and viability of pig oocytes matured in a protein-free medium containing epidermal growth factor, Theriogenology 2000, 54: 787-797, but these have not yet become common techniques. These problems may reflect oocyte cytoplasmic maturation defects that may be due to an incomplete terminal differentiation of the oocytes before meiotic resumption in vitro, Marchal, et al., Effects of cell cycle dependent kinases inhibitor on nuclear and cytoplasmic maturation of porcine oocytes, Mol Reprod Dev 2001, 60: 65-73.

In vivo, meiotically competent oocytes are maintained at the germinal vesicle (GV) stage by the follicular environment until the preovulatory gonadotropin surge. After the preovulatory surge of gonadotrophin, only fully grown and competent oocytes can resume meiosis, complete the first meiotic division, and be ovulated. In contrast, all meiotically competent oocytes matured in vitro spontaneously re-enter the meiotic process as soon as they are removed from their follicles, Edwards RG, Maturation in vitro of mouse, sheep, cow, pig, rhesus monkey and human ovarian oocytes, Nature 1965; 208:349-351. Because investigators have largely failed to produce cloned animals from the meiotically competent oocytes of some species such as the pig, there is obviously a huge difference between a meiotically competent oocyte (i.e. one that is able to resume meiosis upon removal from their follicles), and a developmentally competent oocyte (i.e. one that is able to grow and develop following nuclear transfer and activation). What is needed is a method to preferentially identify and select developmentally competent oocytes from a much larger pool of meiotically competent oocytes.

Work to identify developmentally competent oocytes has generally focused on several limited factors, including the size of follicles selected for collection (Day, et al., In vitro maturation and fertilization of pig oocytes, In: Miller RH, Pursel VG, Norman HD (eds.), Beltsville Symposia in Agricultural Research XX. Biotechnology's Role in the Genetic Improvement of Farm Animals. Savoy, IL: American Society of Animal Science; 1996, 125-144, (about 2-6 mm diameter preferred for porcine follicles)), the size of the oocytes when aspirated from the follicles (Hirao, et al., In vitro growth and maturation of pig oocytes, J Reprod Fertil 1994, 100: 333-339 (110 mm or greater porcine oocytes preferred)), and the various stages of meiosis at which an oocyte can be employed (Stice, U.S. Patent Applicant No. 2001/0053550). Some have also argued that the asynchronous progression of oocytes within the cell cycle detracts from developmental competence.

The time period for maturing oocytes in vitro has also been considered a significant factor in the developmental competence of oocytes. In particular, investigators have conventionally matured a batch of oocytes until most of the oocytes have reached MII in the meiotic cell cycle. Thus, porcine oocytes are commonly matured in vitro for 40-44 hours before manipulation, Betthauser, et al. (2000) (supra); Tao, et al., (1999) (supra); Tao, et al., (1999) (supra); Miyoshi, et al., (2000) (supra); Miyoshi, et al., (2000) (supra); Koo, et al., (2000) (supra); Kuhholzer, et al., (2000) (supra); Uhm, et al., (2000) (supra); Uhm, et al., (2000) (supra); Koo, et al., (2001). In two studies, it was reported that the maturation period of recipient oocytes affects in vitro development of reconstructed embryos in the pig. In these two studies, the blastocyst formation rate of embryos reconstituted with 33 hour matured oocytes was higher than that of embryos reconstituted with 44 hour matured oocytes, Cheong, et al., Development of reconstituted pig embryos by nuclear transfer of cultured cumulus cells, Reprod Fertil Dev 2000, 12: 15-20; Ikeda, et al., Effects of maturational age of porcine oocytes on the induction of activation and development in vitro following somatic cell nuclear transfer, J Vet Med Sci 2001, 63: 1003-1008. In these studies, embryos reconstituted with 33 and 44 hour matured oocytes were activated at 39-40 and 50-51 hours post-initiation of maturation, respectively. Developmental rate to the blastocyst stage after activation of 40 hour matured oocytes was higher than that of 51 hour matured oocytes, and the authors hypothesized that the difference between nuclear transfer groups was due to oocyte activation responses (young vs. old oocytes), Ikeda, et al., (2001) (supra).

Several patent publications have also referred to the significance of in vitro maturation periods to the developmental competence of the oocyte. For example, Campbell, et al. in U.S. Pat. No. 6,252,133, state that “In practice, it is best to enucleate and conduct the transfer as soon as possible after the oocyte reaches metaphase II. The time that this will be post onset of maturation (in vitro) or hormone treatment (in vivo) will depend on the species. For cattle or sheep, nuclear transfer should preferably take place within 24 hours; for pigs, within 48 hours; mice, within 12 hours; and rabbits within 20-24 hours. Although transfer can take place later, it becomes progressively more difficult to achieve as the oocyte ages.” The reference describes fairly lengthy maturation periods (up to 48 hours for porcine), and does not describe how to preferentially select developmentally competent from the culture of oocytes.

Stice, et al. in U.S. Pat. No. 6,235,969, when discussing optimum maturation periods for porcine oocytes, states that “In the present invention, the recipient oocytes will preferably be enucleated at a time ranging from about 30 hours to about 50 hours after the initiation of in vitro maturation, more preferably from about 38 hours to about 42 hours after initiation of in vitro maturation, and most preferably about 40 hours after initiation of in vitro maturation.” Once again, the reference describes fairly lengthy maturation periods (preferably about 40 hours for porcine), and does not describe how to preferentially select developmentally competent from the culture of oocytes.

DeSousa, et al. in PCT Publication No. WO 00/22098, indicate that a porcine oocyte can be optimized for developmental competence by “culturing a harvested oocyte in vitro for between about 36 to about 48 hours.” The reference is specifically directed toward deriving oocytes that are optimized for developmental competence, similarly describes a lengthy maturation process, and also fails to distinguish between developmentally competent and non-developmentally competent embryos within the 36-48 hour cell culture.

Damiani, et al., in U.S. Pat. No. 6,258,998, in discussing the maturation periods for pigs, state that “oocytes can be matured for any period of time: an oocyte can be matured for greater than 10 hours, greater than 20 hours, greater than 24 hours, greater than 60 hours, greater than 72 hours, greater than 90 hours, preferably matured for greater than 36 hours, more preferably matured for greater than 48 hours, and most preferably matured for greater than 53 hours.” While the reference suggests that oocytes matured for fewer hours can be employed in the nuclear transfer process, it expresses a clear preference for oocytes matured for longer periods of time. Moreover, the reference merely identifies a lower bound for the time frame, and does not identify an upper bound. Therefore, the reference does not suggest a maximum time period for oocyte maturation to best assure developmental competence.

### OBJECTS OF THE INVENTION

It is an object of the present invention to provide methods of preferentially identifying developmentally competent oocytes from a culture of meiotically competent oocytes.

It is another object of the invention to provide methods of preferentially identifying developmentally competent oocytes based upon the time required for the oocytes to reach maturation.

Yet another object is to provide a better understanding of the in vivo prophase I maturation process, and the significance of that process to developmental competence.

Still another object of the invention is to improve efficiencies associated with the production of embryos, fetuses, and animals from nuclear transfer cloning procedures.

Another object of the present invention is to provide improved animal livestock breeding programs based upon the improved availability of genetic cloned breeding stock.

Still another object of the present invention is to improve transgenic applications of nuclear transfer technologies by improving the efficiencies associated therewith.

### SUMMARY OF THE INVENTION

The inventors have surprisingly discovered that the optimum maturation period of a recipient oocyte employed in nuclear transfer is much less than conventional teachings actually suggest. In particular, the inventors have determined that oocytes that mature fastest (or soonest) in vitro are typically most developmentally competent to support mitogenesis and growth into a live embryo or animal. It is hypothesized that these “rapidly matured oocytes” are more developmentally competent than their less rapidly matured counterparts because of in vivo follicular maturation that they experience during prophase I of the meiotic cell cycle. For porcine oocytes, the optimum development period has been determined to be less than 30 hours. This is contrary to a consistent line of prior art teachings which suggest that in vitro maturation periods should be maximized.

The inventors have also unexpectedly discovered that the aging of oocytcs in vitro after they have matured to metaphase II does not play a significant role in the ability of the oocytes to support mitogenesis and further development following nuclear transfer. Within reasonable time frames, the fact that an oocyte is aged in vitro after reaching maturation is not very significant; the important issue when selecting oocytes for nuclear transfer is whether the oocyte reached maturation rapidly. Again, this is contrary to a line of prior art teachings that oocytes should not be allowed to age for a significant period of time beyond their maturation point.

Therefore, the key to distinguishing developmentally competent oocyte from mere meiotically competent oocytes is to distinguish rapidly matured oocytes in a culture medium from non-rapidly matured oocytes, and to preferentially select these oocytes for subsequent nuclear transfer. These rapidly matured oocytes can be preferentially selected in a variety of ways. For example, they may be preferentially selected by monitoring the maturation process and selecting the oocytes from the maturation medium that reach maturity soonest. Alternatively, the rapidly matured oocytes can be selected by maturing a culture of oocytes for a shorter time frame than conventionally employed (i.e. a rapid maturation period), and selecting oocytes for nuclear transfer from the culture that reach maturity within that rapid maturation period.

Thus, in one embodiment the invention provides a method of producing a cloned or genetically modified non-human mammalian embryo comprising:

a) providing a cell culture comprising a plurality of in vitro matured oocytes;

b) preferentially selecting a rapidly matured oocyte from the culture;

c) transferring DNA from a donor cell derived from non-human mammalian tissue to the rapidly matured oocyte to form an embryo, fetus, or live animal.

It is hypothesized that these rapidly matured oocytes are better suited for the nuclear transfer process because they have attained a certain level of development competence in vivo in the follicular environment that is not replicated in a cell culture. In particular, the inventors hypothesize that oocytes should surpass the GV-II stage before follicular aspiration in order to ensure optimum developmental competence. This hypothesis supports several additional embodiments of the present invention. For example, in one embodiment histochemical analyses are employed to preferentially select for oocytes that rapidly mature. The rapidly matured oocytes may be identified and preferentially selected based upon histochemical observations made at the time of follicular aspiration or any time thereafter. In another embodiment oocytes that have progressed beyond GV-II in vivo are identified by whether their meiosis is arrested by cell cycle inhibitors such as dbcAMP that preferentially arrest oocytes that are in GV-II. In yet another particular embodiment, the oocytes are matured for a time period that is less than the time required to progress from GV-II to maturation. Again, this is contrary to conventional teachings that oocytes should be matured for longer periods of time to assure optimum developmental competence.

### DISCUSSION

This invention pertains generally to the use of rapidly matured oocytes in the nuclear transfer process, and to improvements in the efficiency of embryo production and animal reproduction obtained by preferentially selecting rapidly matured oocytes for nuclear transfer and cloning operations. Therefore, in one embodiment the invention provides a method of producing a cloned or genetically modified non-human mammalian embryo comprising:

a) providing a cell culture comprising a plurality of in vitro matured oocytes;

b) preferentially selecting a rapidly matured oocyte from the cell culture; and

c) transferring DNA from a donor cell derived from non-human mammalian tissue to the rapidly matured oocyte to form an embryo, fetus, or live animal.

The term “rapidly matured oocyte” is generally synonymous with high levels of developmental competence, but is applied herein to reflect the fact that developmentally competent oocytes can most practically be identified by their rapid maturation rate. This is because, the inventors have unexpectedly discovered, developmentally competent embryos develop sooner in vitro than their non-developmentally competent counterparts. Developmental competence in one embodiment refers to the ability of an oocyte to undergo parthenogenetic activation and develop at least to the morula or to the blastocyst stage in terms of embryo development. In another embodiment developmental competence refers to the ability of an oocyte further to develop into a fetus when implanted into the uterus of a surrogate mother, as characterized by the presence of a genital ridge. In still another embodiment, developmental competence refers to the ability of the fetus further to develop in the uterus and to be birthed as a live cloned animal.

Because aging of the oocytes post-maturation is not an especially significant factor in developmental competence, the selected oocytes can be aged post-maturation for up to 2, 5, 8, 10, 15, or even 20 hours or more post-maturation.

It will be understood than there are numerous other obstacles to the production of cloned embryos and animals that stand in the way of successfully reaching one of these developmental stages. Nevertheless, the use of developmentally competent oocytes in the nuclear transfer process, and more importantly the preferential selection of developmentally competent oocytes over non-developmentally competent oocytes in the nuclear transfer process, ultimately results in higher efficiencies and success in the production of cloned embryos, cell lines and animals. When developmental competence is adjudged by the ability of the parthenogenically activated oocyte to reach the blastocyst stage, one preferably attains numbers of blastocyst stage embryos 5%, 10%, 15%, 20%, 25%, 30%, 35%, 40%, 45%, 50%, 55%, 60%, 65%, 70%, or 75% greater than the numbers of blastocyst stage embryos attained when developmentally competent embryos are not preferentially selected, and developmentally competent and non-developmentally competent matured oocytes are both employed in the procedure (based on all oocytes in culture to reach maturation, and each oocyte being manipulated by nuclear transfer shortly after reaching maturation). An upper limit can be affixed to any of the foregoing percentages equaling 5%, 10%, 25%, 20%, 25%, 30%, 35%, 40%, 45%, or 50% greater (on an additive basis, but only to the extent mathematically feasible) than any of the recited percentages. Thus, for example, one set of ranges in which 70% forms the lower bound is 70%-75%, 70%-80%, 70%-85%, 70%-90%, 70%-95%, and 70%-100%, but the set of ranges does not include 70%-105% because such a range is mathematically impossible.

A more readily applied measure of the improved efficiencies attained in the practice of the instant invention relates to the percentage of nuclear transfer units that reach a particular stage during culture. In absolute terms, in a particularly preferred embodiment at least 8%, 10%, 12%, or 14% of the nuclear transfer units produced in a nuclear transfer procedure and up to about 20%, 30%, 40%, or 50%, reach the blastocyst stage upon further culture for about 7 days. A probability of P<0.05 is preferably associated with the foregoing percentages.

A rapidly matured oocyte can be preferentially selected in a number of ways, to reflect either the increased likelihood of selecting developmentally competent embryos from a plurality of oocytes in culture, or the time period that most accurately reflects the shorter time required for a developmentally competent oocyte to reach maturation. Thus, for example, in one embodiment a rapidly matured oocyte is preferentially selected because of its ability to reach maturation in a culture medium sooner than other oocytes in the culture medium. Therefore, in one series of alternative embodiments, the rapidly matured oocyte is preferentially selected because it reaches maturity sooner than at least 25%, 40%, 50%, 60%, 70%, 80%, 90%, or 95% of the oocytes in culture (based upon the number of oocytes that do in fact reach maturation within about 80 hours). Once again, an upper limit can be affixed to any of the foregoing percentages equaling 5%, 10%, 25%, 20%, 25%, 30%, 35%,40%, 45%, or 50% greater (on an additive basis, but only to the extent mathematically feasible) than any of the recited percentages.

Rapidly matured oocytes can also be preferentially selected based upon the time that the oocytes take to attain maturity in vitro, although it will generally be understood that optimum maturation periods will vary from species to species and that maturation conditions can influence the rate of maturation. Therefore, in one series of preferred embodiments, rapidly matured oocytes are preferentially selected that mature in vitro in an optimum maturation period, which generally in a conventional maturation medium is about 6-52 hours, 10-52 hours, 14-52 hours, 16-52 hours, 20-52 hours, 24-52 hours, 28-52 hours, 32-52 hours, 36-52 hours, 40-52 hours, 44-52 hours, 48-52 hours, 6-48 hours, 10-48 hours, 14-48 hours, 16-48 hours, 20-48 hours, 24-48 hours, 28-48 hours, 32-48 hours, 36-48 hours, 40-48 hours, 44-48 hours, 6-44 hours, 10-44 hours, 14-44 hours, 16-44 hours, 20-44 hours, 24-44 hours, 28-44 hours, 32-44 hours, 36-44 hours, 40-44 hours, 6-40 hours, 10-40 hours, 14-40 hours, 16-40 hours, 20-40 hours, 24-40 hours, 28-40 hours, 32-40 hours, 36-40 hours, 6-36 hours, 10-36 hours, 14-36 hours, 16-36 hours, 20-36 hours, 24-36 hours, 28-36 hours, 32-36 hours, 6-32 hours, 10-32 hours, 14-32 hours, 16-32 hours, 20-32 hours, 24-32 hours, 28-32 hours, 6-28 hours, 10-28 hours, 14-28 hours, 16-28 hours, 20-28 hours, 24-28 hours, 6-24 hours, 10-24 hours, 14-24 hours, 16-24 hours, 20-24 hours, 6-20 hours, 10-20 hours, 14-20 hours, 16-20 hours, 6-16 hours, 10-16 hours, or 6-12 hours. These term “optimum maturation period” refers to the time period required for the oocyte to reach maturation without the influence of naturally or artificially induced arrest, and it will be understood that in practice these numbers will vary if natural or artificial arrested oocytes are employed.

In one series of embodiments, a porcine oocyte is preferentially selected that is matured in vitro in about 6-36 hours, 10-36 hours, 14-36 hours, 16-36 hours, 20-36 hours, 24-36 hours, 28-36 hours, 32-36 hours, 6-32 hours, 10-32 hours, 14-32 hours, 16-32 hours, 20-32 hours, 24-32 hours, 28-32 hours, 6-30 hours, 10-30 hours, 14-30 hours, 16-30 hours, 20-30 hours, 24-30 hours, 28-30 hours, 6-28 hours, 10-28 hours, 14-28 hours, 16-28 hours, 20-28 hours, 24-28 hours, 6-24 hours, 10-24 hours, 14-24 hours, 16-24 hours, 20-24 hours, 6-20 hours, 10-20 hours, 14-20 hours, 16-20 hours, 6-16 hours, 10-16 hours, or 6-12 hours. Particularly preferred porcine oocyte maturation periods are those which range from greater than about 20 hours to less than about 28 hours.

In another series of embodiment a bovine oocyte is preferably selected that is matured in vitro in a time frame of about 4-48 hours, 6-48 hours, 10-48 hours, 14-48 hours, 16-48 hours, 20-48 hours, 24-48 hours, 28-48 hours, 32-48 hours, 36-48 hours, 40-48 hours, 44-48 hours, 4-44 hours, 6-44 hours, 10-44 hours, 14-44 hours, 16-44 hours, 20-44 hours, 24-44 hours, 28-44 hours, 32-44 hours, 36-44 hours, 40-44 hours, 4-40 hours, 6-40 hours, 10-40 hours, 14-40 hours, 16-40 hours, 20-40 hours, 24-40 hours, 28-40 hours, 32-40 hours, 36-40 hours, 4-36 hours, 6-36 hours, 10-36 hours, 14-36 hours, 16-36 hours, 20-36 hours, 24-36 hours, 28-36 hours, 32-36 hours, 4-32 hours, 6-32 hours, 10-32 hours, 14-32 hours, 16-32 hours, 20-32 hours, 24-32 hours, 28-32 hours, 4-28 hours, 6-28 hours, 10-28 hours, 14-28 hours, 16-28 hours, 20-28 hours, 24-28 hours, 4-24 hours, 6-24 hours, 10-24 hours, 14-24 hours, 16-24 hours, 20-24 hours, 4-20 hours, 6-20 hours, 10-20 hours, 14-20 hours, 16-20 hours, 4-16 hours, 6-16 hours, 10-16 hours, 4-12 hours, or 6-12 hours, most preferably about 10-16 hours.

As a practical matter, rapidly matured oocytes can often be identified from a culture that contains multiple oocytes in one of two ways. First, they can be identified by monitoring the maturation process and withdrawing the oocytes in real time that reach maturity rapidly or soonest. Second, they can be identified by maturing cultures of oocytes for shorter time frames than conventionally employed, and selecting the oocytes for nuclear transfer that reach maturity within that shorter time frame. A time period that is shorter than conventionally employed is referred to herein as a “rapid maturation period.” Therefore, when a rapid maturation period is employed to preferentially select rapidly matured oocytes, the invention is characterized as providing a method of producing a cloned or genetically modified non-human mammalian embryo comprising:

a) providing a cell culture comprising one or more oocytes;

b) maturing said one or more oocytes for a rapid maturation period;

c) at the end of the rapid maturation period, selecting a matured oocyte from the cell culture; and

d) transferring DNA from a non-human donor cell to the matured oocyte to form an embryo.

Because of the fact that developmentally competent oocytes reach maturation in vitro sooner than their non-developmentally competent counterparts, the term “rapid maturation period” is generally synonymous with the period of time required for a developmentally competent embryo to reach maturity. A “rapid maturation period” can be characterized in one of several ways, to reflect either the increased likelihood of selecting a developmentally competent oocyte from a culture medium, or the absolute time period for a developmentally competent oocyte to reach maturation. For example, in one embodiment, the term refers to the time required for a less than substantial proportion of the oocytes within a culture medium to reach maturation. A less than substantial proportion of the oocytes in one series of alternative embodiments means less than 75%, 60%, 50%, 40%, 30%, 20%, 10%, or 5% of the oocytes in culture (based upon the number of oocytes that do in fact reach maturation within about 80 hours). Of course, a lower limit can be affixed to any of the foregoing percentages equaling 5%, 10%, 25%, 20%, 25%, 30%, 35%, 40%, 45%, or 50% less (on an additive basis, but only to the extent mathematically feasible) than any of the recited percentages.

The “rapid maturation period” can also be characterized by an absolute time period, based upon the optimum maturation time period observed for a particular species. By selectively withdrawing matured oocytes at the end of the rapid maturation period, one can leave behind the less developmentally competent oocytes that reach maturation at a later time. The rapid maturation period approximates or equals the optimum maturation period discussed in greater detail above for preferentially selecting rapidly matured oocytes.

It is believed that rapidly matured oocytes are better suited for the nuclear transfer process because they have reached a level of development competence during in vivo follicular development that is not generally attainable in cell culture maturation media after spontaneous resumption of meiosis. In particular, it is believed that oocytes that have progressed beyond the GV-II stage before being matured in vitro display significantly greater levels of developmental competence than do oocytes that have not progressed beyond the GV-II stage. Because every oocyte must progress through all four germinal vesicle breakdown stages (GV-I, GV-II, GV-III, and GV-IV) before exiting prophase I and resuming meiosis, and because it takes time to progress through those stages, developmentally competent embryos that have progressed beyond GV-II in vivo can generally be generally identified simply by the time required for them to reach maturation at MII, though it will be understood that the most developmentally competent oocytes are likely to proceed through the germinal vesicle stages more rapidly than less developmentally competent oocytes.

Germinal vesicle stage II refers to the dictyate stage of prophase, and is characterized by a nucleolus and chromatin as a ring or horseshoe around the nucleolus but with some well-stained clumps localized mainly around the ring or horseshoe-like chromatin. Germinal Vesicle Stages I-IV are generally known to workers of skill in the art. Motlik and Fulka, Breakdown of the germinal vesicle in pig oocytes in vivo and in vitro, J Exp. Zool 1976, 198:155-162; Funahashi et al., Synchronization of meiosis in porcine oocytes by exposure to dibutyryl cyclic adenosine monophosphate improves developmental competence following in vitro fertilization, Biol Reprod 1997, 57:49-53.

The time required for an oocyte to progress from GV-II to maturation varies from species to species, but is referred to herein as the post-GV-II maturation period. Because it is believed that oocytes that have progressed beyond GV-II exhibit optimum development competence, in one particular embodiment the rapid maturation period is less than the post-GV-II maturation period. In another preferred embodiment the rapid maturation period approximates or is less than the post-GV-III maturation period. In still another preferred embodiment the rapid maturation period approximates or is less than the post-GV-IV maturation period. Post-GV-II, post-GV-II, and post-GV-IV maturation periods for various species are known generally in the art, and are published in widely circulated journal articles. Sirard et al., Timing of nuclear progression and protein synthesis necessary for meiotic maturation of bovine oocytes, Biol Reprod 1989, 40:1257-1263; Wehrend and Weinecke, Kinetics of meiotic progression, M-phase promoting factor (MPF) and mitogen-activated protein kinase (MAP kinase) activities during in vitro maturation of porcine and bovine oocytes: species specific differences in the length of the meiotic stages, Anim Reprod Sci 2001, 66:175-184. These periods typically mirror the optimum maturation periods discussed in greater detail elsewhere in this document.

Similarly, a rapidly developed oocyte can be equated to an oocyte that has progressed beyond GV-II in vivo (herein referred to as a “post-GV-II in vivo matured oocyte”), to or beyond GV-III in vivo (herein referred to as a “GV-III in vivo matured oocyte”), or to or beyond GV-IV in vivo (herein referred to as a “GV-IV” in vivo matured oocyte”). Histological methods of identifying such oocytes is described in greater detail elsewhere herein.

In yet another embodiment the invention is characterized as a method of producing a cloned or genetically modified non-human embryo by using oocytes that reach developmental competence in vivo. In this embodiment the method comprises:

a) providing a cell culture comprising a plurality of vitro matured oocytes;

b) preferentially selecting a matured oocyte from the culture that attained developmental competence in vivo;

c) transferring DNA from a donor cell derived from non-human mammalian tissue to the matured oocyte to form an embryo.

In still another embodiment the invention provides a method of producing a cloned or genetically modified non-human embryo by culturing oocytes for the time in which a developmentally competent oocyte would achieve maturation. In this embodiment the method comprises:

a) providing a cell culture comprising one or more oocytes that have been matured in vitro for a period of time approximating or less than the time required of a developmentally competent oocyte to reach maturation in vitro;

b) selecting a matured oocyte from the cell culture; and

c) transferring DNA from a donor cell derived from non-human mammalian tissue to the matured oocyte to form an embryo.

As discussed above, in vivo-derived developmental competence can be assessed in a number of ways. Thus, for example, developmental competence can be assessed by the time required for the oocyte to reach a state of maturation. Similarly, developmental competence can be assessed by determining whether the oocyte has progressed in vivo beyond GV-II, or to GV-III or GV-IV. In any of these methods, in vivo developmental competence is preferably reached without the external influence of oocyte maturation protocols and drugs, such as injections of gonadotropin to female species.

It has also surprisingly been discovered that the oocytes preferred for nuclear transfer according to the methods of the current invention (whether termed rapidly matured oocytes, post-GV-II in vivo matured oocytes, or developmentally competent oocytes), can be identified by the fact that they mature in an in vitro maturation medium despite the presence of cell cycle inhibitors, especially those such as dbcAMP that selectively arrest only oocytes that have not progressed beyond GV-II in vivo. I.e., by culturing a plurality of oocytes in the presence of a cell cycle inhibitor, one can distinguish preferred oocytes from non-preferred oocytes simply by determining whether the oocytes reach maturation at all. Thus, in still another embodiment the invention provides a method of producing a cloned or genetically modified non-human embryo comprising:

a) providing a cell culture comprising one or more oocytes and a cell cycle inhibitor;

b) selecting a matured oocyte from the cell culture; and

c) transferring DNA from a non-human donor cell to the matured oocyte to form an embryo.

As can be seen from the foregoing discussion, the optimum maturation period for a particular species can be determined in numerous ways, including:

conducting a series of nuclear transfer experiments employing oocytes that reach maturation at different periods of time;

determining from the literature or experimentally the time required for a post-GV-II in vivo matured oocyte to reach maturity in vitro; and

culturing oocytes in a maturation medium that contains a cell cycle inhibitor such as dbcAMP, identifying the oocytes that reach maturity despite the presence of the cell cycle inhibitor, and determining when those oocytes reach maturity.

Moreover, it will be understood that one or more of the methods described herein to define the preferred oocytes of the instant invention can be combined to further define the preferred oocytes of the instant invention. Thus, for example, rapidly matured oocyte can be identified by the fact that they provide greater nuclear transfer efficiencies, they mature faster than other oocytes in a particular culture medium, and/or they reach maturation within a particular time period.

In still further embodiments any of the foregoing methods can be further carried out by transferring said embryo into a recipient female so as to produce a fetus. In yet another embodiment the fetus undergoes full fetal development and parturition to generate a live-born animal.

Still other embodiments of the instant invention pertain to cloned embryos and animals obtained by the foregoing processes, and to the characterization of those cloned embryos and animals. Thus, in other embodiments, the invention provides a non-human embryo, cell, cell line or animal produced by one of the methods discussed herein.

It will be understood that the descriptor “a” is meant to include plural referents unless the context specifically requires otherwise. Thus, in the practice of this invention, providing a cell culture allows for the provision of multiple cell cultures. Similarly, preferential selection of an oocyte from a cell culture allows for the preferential selection of a plurality of oocytes from the cell culture.

It will be further understood that the invention can be practiced with any mammalian species. The term “mammalian” as used herein refers to any animal of the class Mammalia. The class Mammalia further includes canid (any animal of the family Canidae, including a wolf, a jackal, a fox, or a domestic dog), felid (any animal of the family Felidae, including a lion, a tiger, a leopard, a cheetah,a cougar, or a domestic cat), murid (any animal of the family Muridae including a mouse or a rat), leporid (any animal of the family Leporidae including a rabbit), ursid (any animal of the family Ursidae including a bear), mustelid (any animal of the family Mustelidae including a weasel, a ferret, an otter, a mink, or a skunk), primate (any animal of the Primate order, including an ape, a monkey, a chimpanzee, or a lemur), ungulate (any animal of the polyphyletic group formerly known as the taxon Ungulata, including a camel, a hippopotamus, a horse, a tapir, an elephant, a sheep, a cow, a goat, or a pig), ovid any animal of the fainíly Ovidae (including a sheep), suid any animal of the family Suidae, including a pig or a boar), equid (any animal of the family Equidae, including a zebra an ass, or a horse), bovid (any animal of the family Bovidae, including antelope, an oxen, a cow, a bison, or a goat), caprid (any animal of the family Caprinae, including a goat), and cervid (any animal of the family Cervidae, including a deer). Preferred mammals for practicing the present invention include animals of the class ungulate, ovid, suid, and bovid. Particularly preferred mammalian species for practicing the present invention are bovine and porcine. Porcine species specifically include Large White, Landrace, Meishan, and Minipig.

Nuclear Transfer

In a nuclear transfer procedure, a nuclear donor cell, or the nucleus thereof, is introduced into a recipient cell. Nuclear transfer procedures are known in the literature as described in Campbell, et al., Theriogenology 43 181 (1995); Collas, et al., Mol. Reprod. Dev. 38 264-267 (1994); Keefer, et al., Biol. Reprod. 50 935-939 (1994); Sims, et al., Proc. Nat'l. Acad. Sci. USA 90 6143-6147 (1993); WO-A-9426884; WO-A-9424274; WO-A-9807841; WO-A-9003432; U.S. Pat. No. 4,994,384; and U.S. Pat. No. 5,057,420, each of which is incorporated herein by reference in its entirety.

Oocytes

The term “oocyte” is used to describe the mature animal ovum which is the final product of oogenesis and also the precursor forms being the oogonium, the primary oocyte and the secondary oocyte respectively. Unless otherwise specified herein, the term “oocyte” refers to an unfertilized egg in its natural nucleated state or its enucleated state (i.e., the genetic material that is typically present in the nucleus has been removed). The genetic material typically present in the oocyte nucleus is also referred to herein as maternal genetic material. Maternal genetic material does not include mitochondrial DNA. Unless otherwise specified herein, the term “oocyte” includes oocytes that are either activated or not activated. “Donor genetic material” is the genetic material, obtained from a donor cell, that is introduced into an oocyte. Donor genetic material contains the genetic material that is to be cloned and be present in the cloned non-human mammal. A nuclear transfer embryo or “NT embryo” is the result of introducing donor genetic material into an oocyte, and activating the embryo to induce mitogenesis. Thus, an NT embryo is the nuclear transfer equivalent of a fertilized egg. An NT embryo exists at such time whether or not the maternal genetic material is removed from the oocyte before transfer (i.e., the oocyte is enucleated). A one cell NT embryo is also referred to as a zygote. In some aspects of the present invention, nuclear transfer unit or “NT unit” is produced as a stage that precedes the NT embryo. An “NT unit” is the result of translocating the nuclear material from a donor cell into an oocyte, for instance into the perivitelline space (i.e., the space between an oocyte and the zona pellucida). An NT embryo may contain the maternal genetic material that was originally present in the oocyte.

Typically, oocytes are obtained from the ovaries or reproductive tract of a mammal. Slaughterhouse materials provide a readily available source of oocytes. Alternatively, oocytes can be surgically removed and used in the methods of the present invention. Methods for isolation of oocytes are well known in the art. For instance, the collection of immature bovine oocytes is described by Wells, et al. (Biol. Reprod., 60, 996-1005 (1999 )), and collection of immature porcine oocytes is described by Abeydeera, et al. (Zygote 7, 203-10 (1999)) and Stice, et al., (U.S. Pat. No. 5,945,577). Whole oocytes or bisected oocytes can be used in the present methods. Preferably whole oocytes are used.

Bovine and porcine oocytes are preferably from about 130 to about 230 microns in diameter when aspirated from the follicle. In more preferred embodiments, the oocytes are from about 150 to about 200 microns, and most preferably are about 180 microns in diameter.

Oocytes may be isolated from ovarian follicles at any stage of development, including primordial follicles, primary follicles, secondary follicles, growing follicles, vesicular follicles, maturing follicles, mature follicles, and graafian follicles. The selection of oocytes from porcine ovaries is carried out manually from follicles which are preferably at least about 2 mm in size, and more preferably about 3-8 mm in size. Materials and methods for isolating oocytes from various developmental stages of ovarian follicles are known to those skilled in the art. See, e.g., Laboratory Production of Cattle Embryos, 1994, Ian Gordon, CAB International; Anatomy and Physiology of Farm Animals (5th ed.), 1992, R. D. Frandson and T. L. Spurgeon, Lea & Febiger. In practice, a cumulus oocyte complex (COC) is aspirated from a follicle and the COC is subsequently matured in vitro. Alternatively, in vivo derived oocytes are stripped of their cumulus cells immediately after collection from the donor animals and used in the methods of the present invention. Methods for removing cumulus cells are known to the art (Tao, et al., Anim. Reprod. Sci., 56, 133-41 (1999); Stice, et al., (U.S. Pat. No. 5,945,577). Prior to use, the stage of meiosis of the oocytes is determined using methods known to the art.

In vitro derived oocytes are initially collected from an animal, typically by aspiration of ovarian follicles, while the oocytes are immature. An immature oocyte is an oocyte that is in prophase. Typically, immature oocytes are subsequently cultured in media and allowed to mature under in vitro conditions. Media that can be used for the in vitro maturation of oocytes are referred to herein as maturation media or in vitro maturation (IVM) medium. Examples include Tissue Culture Medium-199 (TCM-199), Waymouths, and NCSU-23 (described in Abeydeera, et al., (Zygote 7, 203-10 (1999)). Preferably TCM-199 is used for cows and NCSU-23 or TCM-199 is used for pigs. The in vitro maturation of oocytes is known to the art. (See, e.g., Prather, et al., Differentiation, 48: 1-8, 1991; Wang, et al., (J. Reprod. Fertil. 111 101 -108 (1997)).

A variety of other media well known to a person of ordinary skill in the art can be used for maturing oocytes in vitro. See, e.g., (i) Alm & Hinrichs, 1996, J. Reprod. Fert. 107: 215-220 and Alm & Torner, 1994, Theriogenology 42: 345-349 for equine oocytes; (ii) Ledda, et al., 1997, Journal of Reproduction and Fertility 109: 73-78; Byrd, et al., 1997, Theriogenology 47: 857-864; Wilmut, et al., 1997, Nature 385: 810-813; and LeGal, 1996, Theriogenology 45:1177-1 for caprine and ovine oocytes; (iii) Lorenzo, et al., 1996, Journal of Reproduction and Fertility 107: 109-117 and Jelinkova, et al., 1994, Molecular Reproduction and Development 37: 210-215 for leporidine oocytes; (iv) Nickson, et al., 1993, J. Reprod. Fert. (Suppl. 47): 231-240; Yamada, et al., 1993, J. Reprod. Fert. (Suppl. 47): 227-229; and Mahi & Yanagimachi, 1976, Journal of Experimental Zoology 196; 189-196 for canine oocytes; (v) Fukui et al., 1991, Theriogenology 35: 499-512 and Pollard, et al., 1995, Theriogenology 43: 301 for cervidine oocytes; and (vi) Del Campo, et al., 1995, Theriogenology 43:21-30; and Del Campo, et al., 1994, Theriogenology 41:187 for camelid oocytes. Oocytes may be cryopreserved and then thawed before placing the oocytes in maturation medium.

Typically, an oocyte is considered mature when it has reached metaphase II (MIII) of the meiotic cell cycle. However, as explained by Stice, et al. in U.S. Application Pub. No. 2001/0053550, oocytes are also sufficiently mature at metaphase I (MI), and can also be used in the methods of the present invention. When used herein, unless otherwise expressly stated, the term “matured” oocyte refers to an oocyte that has reached mataphase II as determined by accepted morphological characteristics.

A recipient oocyte is preferably, but need not be, enucleated, when the nuclear transfer occurs. An oocyte can also be rendered “functionally enucleated,” for example by ultraviolet irradiation. See, e.g., Bradshaw, et al. (1995), Molecular Reproduction and Development 41: 505-12.

In vivo derived oocytes can be obtained from non-superovulated or superovulated donors. Donors can be induced to superovulate by methods known to the art. For instance, superovulated pig or cow donors can be obtained by treatment with PMSG (pregnant mare serum gonadotrophin) or FSH (follicle stimulating hormone). Preferably, oocytes are obtained from the donor animal when the donor is shortly (about 12 hours) after the onset of estrus. The period of time after the onset of estrus within which the oocytes can be obtained depends on the type of animal and is known to the art. For instance, if the donor animal is a cow or a pig the oocytes are preferably obtained within about 24 hours or about 48 hours of the onset of estrus, respectively.

Donor Cells

Donor genetic material contains the genetic material that is to be introduced into an oocyte and be present in the cloned non-human mammal. Donor genetic material can be isolated from a donor cell, i.e., the cell in which the genetic material is normally present. For instance, a nucleus or metaphase plate may be isolated from the donor cell and then introduced into an oocyte. A metaphase plate is described in further detail hereinbelow. Alternatively and preferably, the donor genetic material is not isolated from the donor cell before the donor genetic material is introduced into an oocyte, i.e., the donor cell itself is introduced into an oocyte, typically by introducing the donor cell into the perivitelline space of an oocyte and then fusing the donor cell with the oocyte as described hereinbelow. Optionally, donor genetic material includes DNA that is genetically engineered or transgenic.

The donor cells used in the methods of the present invention can be undifferentiated or differentiated cells, preferably differentiated. Differentiated mammalian cells are those cells which are beyond the early embryonic stage. More particularly, the differentiated cells are those from at least beyond the embryonic disc stage (for instance, about day 10 of bovine embryogenesis, or about day 8 of pig embryogenesis). Embryogenic stages from at least beyond the embryonic disc stage are referred to herein as late embryogenic stage. Fetal stage cells are those cells that are at least about day 20 to at least about day 30 of embryogenesis up to the time of birth. Adult stage cells are those present in an animal after birth. The differentiated cells may be derived from ectoderm, mesoderm or endoderm; preferably they are derived from mesoderm or endoderm.

Non-human mammalian cells for use as donor cells may be obtained by methods known to the art. Mammalian cells useful in the present invention include cells of the body, including, by way of example, epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells. The mammalian cells that can be used in the methods of the present invention may be obtained from different organs, e.g., skin, lung, pancreas, liver, stomach, intestine, heart, reproductive organs, bladder, kidney, urethra and other urinary organs. The mammalian cells may be somatic or diploid germ cells obtained from embryo, fetus, or adult tissue, or from cultured cell lines, preferably adult tissue. The use of adult cells is advantageous as it allows the cloning of animals having desirable characteristics. These are just examples of suitable cells that can be used as a source of donor genetic material. Preferably, the cells are fibroblasts or granulosa cells.

In one aspect of the invention, the donor cell, whether it is introduced directly into an oocyte or used as a source of a donor nucleus or a donor metaphase plate that is introduced into an oocyte, is a quiescent cell (i.e., a cell at G0, see, for instance, Wilmut, et al., Nature, 385, 810-3 (1997); Campbell, et al., WO 97/07669), a proliferating cell (Stice, et al., U.S. Pat. No. 5,945,577), a metaphase cell, a cell arrested at metaphase, or a cell arrested at late G1 phase. Whether a donor cell is quiescent, proliferating, at metaphase, arrested at metaphase, or arrested at late GI phase can be determined by methods known to the art. For example, a donor cell at metaphase is a cell that has progressed through the cell cycle including the prophase stage of mitosis; the centromeres joining the condensed sister chromatids are present in the region of the equatorial plane of the cell, and the nuclear membrane is absent. The appearance of the chromosomes of a metaphase cell is known to the art and is referred to as the metaphase plate. For example, a donor cell at late GI is a cell that has intracellular concentrations of regulatory proteins, for instance, cyclin A and cyclin E, that are higher than in cells at other cell cycle phases. A donor cell arrested at metaphase or arrested at late G1 phase is unable to proceed beyond metaphase or G1 into anaphase or S phase, respectively, and is therefore no longer proliferating. Quiescent cells are not in any of the four phases of the cell cycle (i.e., G1, S, G2, or M). Quiescent cells are typically considered as being in the G0 state so as to indicate that they would not normally progress through the cycle. The nucleus of a quiescent G0 cell is diploid. Thus, in contrast to a quiescent cell, a cell arrested at metaphase does not have a nucleus, and the DNA content is tetraploid. In contrast to a quiescent cell, a cell arrested at late GI is prepared to undergo DNA replication but is still diploid.

Preferably, a donor cell is quiescent, arrested at G1, at metaphase, or arrested at metaphase. Placing the metaphase donor genetic material into an oocyte is advantageous because it facilitates additional exposure to cytoplasmic reprogramming factors needed for reprogramming donor genetic material that has been introduced into the oocyte. Placing the donor genetic material arrested at late G1 into an oocyte is advantageous because the donor nucleus is prepared to undergo DNA replication during S phase of the first cell cycle of the NT embryo.

Donor cells can be arrested in metaphase by exposing the cells to at least one arresting agent. Useful arresting agents include nocodazole, demicolchin, colchicine, colcemid, paclitaxel, docetaxel, otoposide, vinblastine, vincristine, vinorelbine, monastrol, and taxol, preferably nocodazole. Preferably, the arrested state of the donor cell is reversible, i.e., the cell resumes proliferating when the arresting agent(s) is removed. The exposure of a population of donor cells to an arresting agent typically does not result in arrest of all the donor cells, thus those cells that are arrested (and therefore typically at metaphase) can be separated from those that are not arrested. Cells arrested at metaphase typically have an altered morphology that allows arrested cells to be separated. For instance, arrested cells grown on a surface and then exposed to an arresting agent have a “rounded up” appearance while proliferating cells are relatively flat.

Donor cells may be arrested at GI by exposing the cells to at least one arresting agent. Useful arresting agents include mimosine, aphidocoline, and inhibitors of CDK2 kinase, including for instance roscovitine or olomoucine (see, for instance, Alessi, et al., Exp. Cell Res., 245, 8-18 (1998)). Preferably, roscovitine or olomoucine, more preferably roscovitine, are used, although contact inhibition is another particularly preferred technique. Preferably, the arrested state of the donor cell is reversible, i.e., the cell resumes proliferating when the arresting agent(s) is removed. The exposure of a population of donor cells to an arresting agent typically does not result in arrest of all the donor cells, thus those cells that are arrested (and therefore typically at late G1) can be separated from those that are not arrested. Cells at G1 typically have an altered morphology that allows arrested cells to be separated. For instance, arrested cells are typically smaller in size than those cells that are not arrested at late G1. Preferably, donors cells arrested in late G1 having a size of about 15 μM to about 20 μM in size are selected for introduction into an oocyte. Donor cells can be induced to enter quiescence by employing various conventional methods of inducing quiescence such as serum starvation and contact inhibition (i.e. growth in culture to confluence). A preferred method is contact inhibition.

Donor genetic material can be isolated from quiescent cells, proliferating cells, cells that are at metaphase, cells that are arrested at metaphase, or cells arrested at late G1 using methods known to the art (see, for instance, Collas and Barnes, Mol. Reprod. Dev., 38, 264-267 (1994)). Typically, a donor nucleus can be isolated by removing the cell membrane, or further isolated by removing at least some of the cytoplasm that normally surrounds the donor nucleus.

A variety of methods for culturing donor cells exist in the art. See, e.g., Culture of Animal Cells: a manual of basic techniques (3rd edition), 1994, Freshney (ed.), Wiley-Liss, Inc.; Cells: a laboratory manual (vol. 1), (1998), Spector, Goldman, Leinwand (eds.), Cold Spring Harbor Laboratory Press; and Animal Cells: culture and media, 1994, Darling & Morgan, John Wiley and Sons, Ltd.

Nuclear Translocation

A nuclear donor can be translocated into an oocyte, most preferably an enucleated oocyte, using a variety of materials and methods that are well known to a person of ordinary skill in the art. Isolated donor genetic material may be injected directly into an oocyte to produce the NT embryo (see, for instance, Collas and Barnes, Mol. Reprod. Dev., 38, 264-267 (1994); and Tao, et al., Anim. Reprod. Sci., 56, 133-41 (1999)). A peizo element based micromanipulator may be used to facilitate microinjection tasks (see, for instance, Wakayama, et al., Nature, 394, 369-74 (1998)). It is expected that a nuclear membrane will form around a metaphase plate that is introduced into an oocyte.

Alternatively, a single donor cell of the same species as the oocyte may be introduced by fusing the cell with the oocyte after the donor cell is placed in the perivitelline space of the oocyte (i.e., the space between an oocyte and the zona pellucida) to produce an NT unit. Such methods are known to the art (see, for instance, Stice, et al., (U.S. Pat. No. 5,945,577)). A variety of electrofusion media can be used including e.g., sucrose, mannitol, sorbitol and phosphate buffered solution. Fusion can also be accomplished using Sendai virus as a fusogenic agent (Graham, Wister Inot. Symp. Monogr., 9, 19, 1969), or by using polyethylene glycol (PEG) (Susko-Parrish, et al., U.S. Pat. No. 5,496,720). Other examples of non-electrical means of cell fusion involve incubating in solutions comprising trypsin, dimethylsulfoxide (DMSO), lectins, and agglutinin viruses. Fusion of the donor cell and the oocyte that make up an NT unit result in an NT embryo.

Typically, in electrofusion of porcine oocytes and donor cells, a fusion pulse ranging from about 150 V/mm to about 350 V/mm, more preferably about 250 V/mm, is used. The duration of the pulse may be about 20 μseconds. For electrofusion of bovine oocytes and donor cells, a fusion pulse of about 40 V/150 μm may be used. The duration of the pulse is about 20 μseconds. Multiple pulses can also be used successfully to induce cell fusion.

The nuclear translocation is preferably performed within about 15, 12, 10, 8, 6, 4, or 2 hours of maturation.

NT Embryo

If desired, an NT embryo can be cultured in media. The type of media can depend on the species of oocyte. For instance, for pig cells, NCSU-23 or other pig embryo culture medium (see, for instance, Tao, et al., Anim. Reprod. Sci., 56, 133-41 (1999)) can be used. Preferably, for pig cells, a sequential media system is used. The first medium of the sequential media system is a bicarbonate-buffered culture medium that includes alanine, alanyl-glutamine, asparagine, aspartic acid, calcium chloride, EDTA, glucose, glutamate, glycine, human serum albumin, magnesium sulphate, penicillin G, potassium chloride, proline, serine, sodium bicarbonate, sodium chloride, sodium hydrogen phosphate, sodium lactate, sodium pyruvate, and taurine is used. Such a culture medium is available under the trade designation Gl.2(Vitrolife, Inc., Englewood Colo.). The second medium of the sequential media system is a bicarbonate-buffered culture medium that includes alanine, alanyl-glutamine arginine, asparagine, aspartic acid, calcium chloride, calium pantothenate, choline chloride, cystine, folic acid, glucose, glutamate, glycine, Histidine, human serum albumin, i-inositol, isoleucine, leucine, lysine, magnesium sulphate, methionine, niacinamide, penicillin G, phenylalanine, potassium chloride, proline, pyridoxine, riboflavin, serine, sodium bicarbonate, sodium chloride, sodium Hydrogen phosphate, sodium lactate, sodium pyruvate, thiamine, threonine, tryptophan, tyrosine, valine. Such a culture medium is available under the trade designation G2.2 (Vitrolife, Inc.). This sequential media system is referred to herein as G1/G2, or G1.2/G2.2. For cow cells, G1/G2, KSOM, CR, or TCM-199, can be used. The NT embryo is typically incubated for up to about 10 hours. Preferably, an NT embryo is not incubated so long that the chromosomes begin to disassociate from each other, and/or micronuclei are formed after activation. Alternatively, an NT embryo need not be cultured in media.

If the oocyte used to produce the NT embryo was not enucleated, the NT embryo, whether incubated in medium or not, can optionally be enucleated. Enucleation of an NT embryo involves removal of maternal genetic material from the NT embryo, but not removal of donor genetic material. Enucleation of an NT embryo is discussed hereinbelow. Preferably, when the oocyte used to produce the NT embryo was not enucleated, the method of the invention preferably includes enucleation of the NT embryo. Further, if the oocyte used to produce the NT embryo was not activated, the method preferably includes activation of the NT embryo. Activation of an NT embryo can be performed either before or after the enucleation step.

Enucleation

Oocytes may be enucleated before introduction of donor genetic material. Enucleation of oocytes may be accomplished microsurgically using a micropipette to remove the polar body and the adjacent cytoplasm, or by chemical treatment (see, for instance, Baguisi, et al., Theriol., 53, 290 (2000)). If enucleation is performed prior to introduction of donor genetic material, it may be conducted using methods previously described for enucleating MII oocytes (Tao, et al., Anim. Reprod. Sci., 56, 133-41 (1999)) or by methods such as described by Goto, et al., (Anim. Sci. J, 70, 243-245 (1999)). The oocytes may then be screened to identify those successfully enucleated. This screening can be done by staining the oocytes with a detectable marker that specifically binds to DNA (for instance, 1 μg/ml 33342 Hoechst dye in HEPES buffered hamster embryo culture medium (HECM, Seshagine, et al., Biol. Reprod., 40, 544-606, (1989)), and then viewing under ultraviolet irradiation for less than 10 seconds either the oocytes or the cytoplasm and maternal genetic material removed during the enucleation procedure. The oocytes that have been successfully enucleated can then be placed in a suitable culture medium, e.g., TCM-199, G1/G2, or CR1aa plus 10% serum (Stice, et al., U.S. Pat. No. 5,945,577).

In vitro matured oocytes enucleated before introduction of donor genetic material can be enucleated when they are at the appropriate stage, e.g., immature germinal vesicle, maturing (MI to MII), or mature. In vivo matured oocytes enucleated before introduction of donor genetic material can be enucleated after isolation, preferably immediately after isolation.

If the oocyte used to produce the NT embryo was not enucleated, then the NT embryo can be enucleated. Within the NT embryo, the maternal genetic material can be distinguished from the donor genetic material by, for instance, the position of the donor nucleus within the NT embryo, formation of the first polar body, or a combination thereof. The known location of the donor genetic material within the NT embryo is based on where it was placed in the perivitelline space in relation to the location of the maternal genetic material. The maternal genetic material is near the opening placed in the zona pellucida during transfer of the donor genetic material, preferably the donor genetic material is placed away from that area. Therefore that area of cytoplasm (near the opening in the zona) can be removed via either enucleation pipette or by expulsion of cytoplasm through the opening in the zona, preferably by enucleation pipette (see, e.g., Prather, et al., Biol. Reprod., 37, 859 (1987); and Goto, et al., Anim. Sci. J, 20, 243-245 (1999)). When MI oocytes are employed in the nuclear transfer process, the oocyte may progress in meiosis to MII after introduction of the donor genetic material. If so, then the first polar body can also be used as landmark to find the maternal genetic material. Hoechst dye can be used to visualize genetic material, including confirming the presence of the maternal genetic material in the removed cytoplasm. These methods may be used alone or in conjunction with each other to verify location of chromosomes and verify enucleation of the oocyte.

An NT embryo that contains both maternal and donor genetic material need not be immediately enucleated or, in some aspects of the invention, is not enucleated at all. That is, the NT embryo will at least transiently contain both maternal genetic material and donor genetic material. For instance, Willadsen, et al. (Nature, 320, 63-65 (1986)), used non-enucleated NT embryos derived from MII oocytes to produce cloned sheep embryos. It is expected that maternal genetic material may contribute to only the placenta, thus the cells that develop to eventually form a fetus or offspring would not contain the full complement of maternal genetic material (diploid).

Activation

An oocyte or an NT embryo may be activated using artificial activation methods known to the art (see, for instance, Susko-Parrish, et al., (U.S. Pat. No. 5,496,720); and Stice, et al., (U.S. Pat. No. 5,945,577)). An oocyte may be activated before introduction of donor genetic material, or at the same time as the introduction of donor genetic material. Alternatively and preferably, an NT embryo may be activated. Typically, when an oocyte is activated before introduction of donor genetic material, the activated oocyte is used immediately or within about 10 hours after activation. When an NT embryo is activated, activation is done at about the same time as introduction of the donor genetic material or up to about 10 hours following introduction.

Activation may include the use of agents that decrease protein phosphorylation in the cell, decrease protein synthesis by the cell, or increase the level of cations in the cell. Protein phosphorylation can be decreased by the use of agents that inhibit phosphorylation, including, for instance, a serine-threonine kinase inhibitor like 6-dimethylaminopurine, staurosporine, 2-aminopurine, or sphingosine. Protein phosphorylation can also be decreased by the use of agents that cause dephosphorylation of proteins, including for instance phosphatases A or B. Agents that decrease protein synthesis by the cell include, for instance, cycloheximide. Agents that increase the level of cations in the cell include, for instance, ionomycin, ionophores, ethanol, media free of Mg⁺⁺ and Ca⁺⁺, phorbol esters, and electrical shock. Other agents that can be used include thimerasol and DTT (Machaty, et al, Biol. Reprod., 57, 1123 (1997)).

In a preferred embodiment, activation is achieved by increasing the levels of cations in the cell (preferably calcium ions), and decreasing protein phosphorylation. In another preferred embodiment, activation is achieved by increasing the levels of cations in the cell, and decreasing protein synthesis.

Specific examples of activation methods are listed below.

1. Activation by lonomycin and DMAP: 1 - Place oocytes in lonomycin (5 μM) with 2 mM of DMAP for 4 minutes; 2- Move the oocytes into culture media with 2 mM of DMAP for 4 hours; 3- Rinse four times and place in culture.

2. Activation by lonomycin, DMAP and Roscovitin: 1—Place oocytes in lonomycin (5 μM) with 2 mM of DMAP for four minutes; 2—Move the oocytes into culture media with 2 mM of DMAP and 200 microM of Roscovitin for three hours; 3—Rinse four times and place in culture.

3. Activation by exposure to Ionomycin followed by cytochalasin and cycloheximide: 1—Place oocytes in lonomycin (5 microM) for four minutes; 2—Move oocytes to culture media containing 5 μg/ml of cytochalasin B and 5 μg/ml of cycloheximide for five hours; 3—Rinse four times and place in culture.

4. Activation by electrical pulses: 1—Place eggs in mannitol media containing 100 μM CaCl₂; 2—Deliver three pulses of 1.0 kVcm⁻¹ for 20 μsec, each pulse 22 minutes apart; 3- Move oocytes to culture media containing 5 μg/ml of cytochalasin B for three hours.

5. Activation by exposure with ethanol followed by cytochalasin and cycloheximide: 1—Place oocytes in 7% ethanol for one minute; 2—Move oocytes to culture media containing 5 μg/ml of cytochalasin B and 5 μg/ml of cycloheximide for five hours; 3—Rinse four times and place in culture.

6. Activation by microinjection of adenophostine: 1—Inject oocytes with 10 to 12 picoliters of a solution containing 10 μM of adenophostine; 2—Put oocytes in culture.

7. Activation by microinjection of sperm factor: 1—Inject oocytes with 10 to 12 picoliters of sperm factor isolated, e.g., from primates, pigs, bovine, sheep, goats, horses, mice, rats, rabbits or hamsters; 2—Put eggs in culture.

8. Activation by microinjection of recombinant sperm factor.

9. Activation by Exposure to DMAP followed by Cycloheximide and Cytochalasin B: 1—Place oocytes or NT units in about 2 mM DMAP for about one hour, followed by incubation for about two to twelve hours, preferably about eight hours, in 5 μg/ml of cytochalasin B and 20 μg/ml cycloheximide.

Activation of porcine oocytes and NT embryos may use about 1% to about 20% ETOH, preferably 8% ETOH in KSOM or G1/G2 culture medium for 10 minutes followed by about 1 mM to about 10 mM DMAP, preferably about 2 mM DMAP in KSOM or G1/G2 for 5 hours. Preferably, porcine oocytes and NT embryos are activated by applying two pulses of from about 50 V/mm to about 200 V/mm (direct current), more preferably about 75 V/mm. The two pulses are each preferably about 60 μseconds long, and preferably separated by about a 5 second interval. Preferably, the activation is done in Zimmerman fusion media (Zimmerman, et al., Membrane Biol., 67, 165-182(1982)).

Bovine oocytes and NT embryos may be activated by the method of Yang, et al. (Biol. Reprod., 42(Suppl 1), 117 (1992)), more preferably, by exposing bovine oocytes to about 1 μM to about 100 μM ionomycin, preferably about 50 μM ionomycin, for 10 minutes and about 1 μg/ml to about 100 μg/ml cycloheximide, preferably about 10 μg/ml cycloheximide, for about 2 hours to about 10 hours, preferably about 6 hours. Preferably, bovine oocytes and NT embryos are activated by exposure to agents that increase the level of cations in the cell, followed by exposure to agents that decrease protein synthesis in the cell and/or agents that are microfilament inhibitors. Most preferably, bovine oocytes and NT embryos are exposed to about 1 μM to about 100 μM calcium ionophore, preferably about 5 μM calcium ionophore, for about 10 minutes. This is followed by incubation in about 1 μg/ml to about 10 μg/ml cytochalasin B, preferably about 5 μg/ml cytochalasin B, and about 1 μg/ml to about 100 μg/ml cycloheximide, preferably about 10 μg/ml cycloheximide, for about 1 hour. This is followed by incubation in about 1 μg/ml to about 100 μg/ml cycloheximide, preferably about 10 μg/ml cycloheximide, for about 5 hours. Preferably, after the activation treatments, bovine NT embryos are cultured in BARC medium (Powell, et al., Theriogen., 55, 287 (2001)).

Whether a porcine or bovine oocyte or a porcine or bovine NT embryo has been activated can be determined by observing swelling of the donor nucleus, and cleavage of the embryo about 10 hours to about 30 hours after activation.

Instead of using artificial activation methods, or in conjunction with artificial activation methods, fertilized oocyte cytoplasm can be used to activate an oocyte or an NT embryo. The use of fertilized oocyte cytoplasm to activate an oocyte or an NT embryo is referred to herein as “natural activation.” Fertilized oocyte cytoplasm can be obtained by removal of cytoplasm from an oocyte that has been fertilized by a sperm. Fertilized oocyte cytoplasm can be removed by pipette and then injected directly into the oocyte or NT embryo that is to be activated. It is expected that fertilized oocyte cytoplasm can be injected in volumes up to between about 10% and about 50% the volume of the oocyte or NT embryo that is to be activated.

As noted, activation may be effected before, simultaneous, or after nuclear transfer. In general, activation will be effected about 40 hours prior to nuclear translocation to about 40 hours after nuclear translocation, more preferably about 24 hours before to about 24 hours after nuclear translocation, and still even more preferably from about 4 to 9 hours before nuclear translocation to about 4 to 9 hours after nuclear translocation. Most preferably activation occurs at 0-9 hours, 0-8 hours, 0-7 hours, 0-6 hours, 0-5 hours, 0-4 hours, 0-3 hours, 0-2 hours, or 0-1 hours post-nuclear translocation. Activation is most preferably effected after or proximate to in vitro maturation of the oocyte.

Assessment of Successful Nuclear Reprogramming and Transfer of Activated NT Embryos

Successful nuclear reprogramming is evaluated by determining if activated NT embryos develop to the blastocyst stage. For both pig and cow, development of an activated NT embryo to blastocyst is typically complete in seven days, and typically includes the trophoblast and inner cell mass.

An activated NT embryo may be transferred immediately into a recipient animal or cultured for up to about 8 days in, for instance, KSOM medium, NCSU-23 medium, BARC medium, G1.2/G2.2 culture medium, or others well known to the art (see for instance Stice, et al., U.S. Pat. No. 5,945,577; Wells, et al., Biol. Reprod., 60, 996-1005 (1999 ); and Tao, et al., Anim. Reprod. Sci., 56, 133-41 (1999)). Preferably, an activated NT embryo is cultured for between about 12 hours to about 36 hours (for porcine NT embryos) or for about 7 to about 8 days (for bovine NT embryos). Then, intact NT embryos (some cleaved) are transferred into a synchronous recipient animal, i.e., the transferred NT embryo is at the same stage, or about a day before or a day after, as a fertilized embryo would be in the recipient. For pigs, from about one to about 300 NT embryos can be transferred into each recipient female but typically about 50 to about 150 embryos are transferred and ideally 100 embryos are transferred. Methods of surgical and non-surgical transfer in animals is well known in the art. For instance, surgical and non-surgical transfer in pigs is described by Cumock, et al., (Amer. J Vet. Res., 37, 97-98 (1976 )), and Hazeleger, et al., (Theriogenol., 51, 81-91 (1999)). Preferably, the animal is of the same species as the donor genetic material of the NT embryo.

Ultrasound and non-return to estrus is used to determine which recipients are pregnant. If needed for tissue or cell transplantation NT fetuses can be harvested during the pregnancy through surgical recovery. If live calves or pigs are desired the pregnancy lasts approximately 285 days or 114 days respectively, and some offspring may require neonatal assistance in the form of oxygen supplementation and other interventions (Hill, et al., Theriogenol., 51, 1451 (1999)).

Other Sources of Information

Where descriptions of oocyte maturation, oocyte enucleation, cell activation, in vitro embryo development, and other processes are described herein in relation to mammals in general, the following references provide additional descriptions of such process for specific mammals. The following references are provided to aid the reader in understanding the invention and are not admitted to describe or constitute prior art to the present invention. With regard to suids, researchers have reported materials and methods for oocyte maturation, oocyte enucleation, cell activation, in vitro embryo development, and other processes. See, e.g., Grocholová, et al., 1997, J. Exp. Zoology 277: 49-56; Schoenbeck, et al., 1993, Theriogenology 40: 257-266; Prather, et al., 1989, Biology of Reproduction 41: 414-418; Prather, et al., 1991, Molecular Reproduction and Development 28: 405-409; Jolliff & Prather, 1997, Biol. Reprod. 56:544-548; Mattioli, et al., 1991, Molecular Reproduction and Development 30: 109-125; Terlouw, et al., 1992, Theriogenology 37: 309; Prochazka, et al., 1992, J. Reprod. Fert. 96: 725-734; Funahashi, et al., 1993, Molecular Reproduction and Development 36: 361-367; Prather, et al., Bio. Rep. Vol. 50 Sup 1: 282; Nussbaum, et al., 1995, Molecular Reproduction and Development 41: 70-75; Funahashi, et al., 1995, Zygote 3: 273-281; Wang, et al., 1997, Biology of Reproduction 56: 1376-1382; Piedrahita, et al., 1989, Biology of Reproduction 58: 1321-1329; Macháty, et al., 1997, Biology of Reproduction 57: 85-91; and Machaty, et al., 1995, Biology of Reproduction 52: 753-758.

With regard to bovids, researchers have reported materials and methods for oocyte maturation, oocyte enucleation, cell activation, in vitro embryo development, and other processes. See, e.g., U.S. Pat. Nos. 5,453,357 and 5,670,372, entitled “Pluripotent Embryonic Stem Cells and Methods of Making Same,” Hogan; Sims & First, 1993, Theriogenology 39:313; Keefer, et al., 1994, Mol. Reprod. Dev. 38: 264-268; U.S. Pat. No. 4,994,384, “Multiplying Bovine Embryos,” Prather, et al.; U.S. Pat. No. 5,057,420, “Bovine Nuclear Transplantation,” Massey & Willadsen; Delhaise, et al., 1995, Reprod. Fert. Develop. 7: 1217-1219; Lavoir 1994,J. Reprod. Dev. 37: 413-424; PCT application WO 95/10599 entitled “Embryonic Stem Cell-Like Cells”; Stice, et al., 1996, Biol. Reprod. 54: 100-110; Strelchenko, 1996, Theriogenology 45: 130-141; WO 97/37009, entitled “Cultured Inner Cell Mass Cell-Lines Derived from Ungulate Embryos,” Stice and Golucke, published Oct. 9, 1997; U.S. Pat. No. 5,213,979, entitled “In vitro Culture of Bovine Embryos,” First, et al., May 25, 1993; U.S. Pat. No. 5,096,822, entitled “Bovine Embryo Medium,” Rosenkrans, Jr., et al., Mar. 17, 1992; Seidel and Elsden, 1997, Embryo Transfer in Dairy Cattle, W. D. Hoard & Sons, Co., Hoards Dairyman; Stice & Keefer, 1993, “Multiple generational bovine embryo cloning,” Biology of Reproduction 48: 715-719; Wagoner, et al., 1996, “Functional enucleation of bovine oocytes: effects of centrifugation and ultraviolet light,” Theriogenology 46: 279-284; Pieterse, et al., 1988, “Aspiration of bovine oocytes during transvaginal ultrasound scanning of the ovaries,” Theriogenology 30: 751-762; Saito, et al., 1992, Roux's Arch. Dev. Biol. 201: 134-141; and U.S. Pat. No. 5,496,720, entitled “Parthenogonic Oocyte Activation,” Mar. 5, 1996, Susko-Parrish, et al.

With regard to ovids and caprids, researchers have reported materials and methods for oocyte maturation, oocyte enucleation, cell activation, in vitro embryo development, and other processes. See, e.g., Willadsen, 1986, Nature 320: 63-66; Ruffing, et al., 1993, Biology of Reproduction 48: 889-904; Smith & Wilmut, 1989, Biology of Reproduction 40: 1027-1035; McLaughlin, et al., 1991, Theriogenology 35: 240; Campbell, et al., 1995, Theriogenology 43: 181; Campbell, et al., 1996, Theriogenology 45: 286; Campbell, et al., 1996, Nature 380: 64-66; Wilmut, et al., 1997, Nature 385: 810-813; Ledda, et al., 1997, Journal of Reproduction and Fertility 109:73-78; Byrd, et al., 1997, Theriogenology 47: 857-864; Wilmut, et al., 1997, Nature 385: 810-813; LeGal, 1996, Theriogenology 45: 1177-1; Pawshe, et al., 1996, Theriogenology 46: 971-982; Gall, et al., 1993, Molecular Reproduction and Development 36: 500-506; Walker, et al., 1996, Biology of Reproduction 55: 703-708; and Gardner, et al., 1994, Biology ofReproduction 50: 390-400.

Transgenic Applications

In particularly preferred embodiments, embryos, fetuses and/or animals of the invention are transgenic. The term “transgenic” as used herein refers to an embryo, fetus or animal comprising one or more cells whose genome has been altered using recombinant DNA techniques. In preferred embodiments, a transgenic embryo, fetus, or animal comprises one or more transgenic cells. While germ line transmission is not a requirement of transgenic embryos, fetuses, or animals as that term is used herein, in particularly preferred embodiments a transgenic embryo, fetus, or animal can pass its transgenic characteristic(s) through the germ line. In certain embodiments, a transgenic embryo, fetus or animal expresses one or more exogenous genes, as exogenous RNA and protein molecules. Most preferably, a transgenic embryo, fetus or animal results from a nuclear transfer procedure using a transgenic nuclear donor cell.

Materials and methods readily available to a person of ordinary skill in the art can be utilized to convert the nuclear donor cells of the invention into transgenic cells. Once nuclear DNA is modified in a nuclear donor cell, embryos, fetuses, and animals arising from these cells can also comprise the modified nuclear DNA. Hence, materials and methods readily available to a person of ordinary skill in the art can be applied to nuclear donor cells to produce transgenic cloned and chimeric animals. See, e.g., EPO 254 166, entitled “Transgenic Animals Secreting Desired Proteins Into Milk;” WO 94/19935, entitled “Isolation of Components of Interest From Milk;” WO 93/22432, entitled “Method for Identifying Transgenic Pre-implantation Embryos;” WO 95/17085, entitled “Transgenic Production of Antibodies in Milk;” Hammer, et al., 1985, Nature 315: 5.80-685; Miller, etal., 1986,J. Endocrinology 120: 481488; Williams, et al., 1992, J Ani. Sci. 70: 2207-2111; Piedrahita, et al., 1998, Biol. Reprod. 58: 1321-1329; Piedrahita, et al., 1997, J Reprod. Fert. (suppl.) 52: 245-254; and Nottle, et al, 1997, J. Reprod. Fert. (suppl.) 52: 245-254, each of which is incorporated herein by reference in its entirety including all figures, drawings and tables.

Methods for generating transgenic cells typically include (1) assembling a suitable DNA construct useful for inserting a specific DNA sequence into nuclear: DNA of a cell; (2) transfecting the DNA sequence into cells; (3) allowing random insertion and/or homologous recombination to occur. A modification resulting from such a process may include insertion of a suitable DNA construct(s) into a target genome; deletion of DNA from a target genome; and/or mutation of a target genome.

DNA constructs can comprise a gene of interest as well as a variety of elements including regulatory promoters, insulators, enhancers, and repressors as well as elements for ribosomal binding to RNA transcribed from a DNA construct. DNA constructs can also encode ribozymes and anti-sense DNA and/or RNA. Moreover, DNA constructs can comprise a selection element, such as a gene for drug selection of transformants. These examples are well known to a person of ordinary skill in the art and are not meant to be limiting.

Due to effective recombinant DNA techniques available in conjunction with DNA sequences for regulatory elements and genes readily available in data bases and the commercial sector, a person of ordinary skill in the art can readily generate a DNA construct appropriate for establishing transgenic cells using materials and methods described herein. For example, transfection techniques are well known to a person of ordinary skill in the art and materials and methods for carrying out transfection of DNA constructs into cells are commercially available. For example, materials that can be used to transfect cells with DNA constructs are lipophillic compounds, such as Lipofectin™, Superfect™, LipoTAXI™, and CLONfectin™. Particular lipophillic compounds can be induced to form liposomes for mediating transfection of the DNA construct into the cells. In addition, cationic based transfection agents that are known in the art can be utilized to transfect cells with nucleic acid molecules (e.g., calcium phosphate precipitation, DEAE-dextran, polybrene, polyamine). Other techniques are known in the art that use protein-based or amphipathic polyamines as transfection reagents. Also, electroportation techniques known in the art can be utilized to translocate nucleic acid molecules into cells. Particle bombardment techniques are also known in the art for introducing exogenous DNA into cells.

Target sequences from a DNA construct can be inserted into specific regions of nuclear DNA by rational design of a DNA construct. Such design techniques and methods are well known to a person of ordinary skill in the art. See, U.S. Pat. No. 5,633,067, “Method of Producing a Transgenic Bovine or Transgenic Bovine Embryo;” DeBoer, et al., issued May 27, 1997; U.S. Pat. No. 5,512,205, “Homologous Recombination in Mammalian Cells;” Kay et al., issued Mar. 18, 1997; and PCT publication WO 93/22432, “Method for Identifying Transgenic Pre-Implantation Embryos,” each of which is incorporated herein by reference in its entirety, including all figures, drawings, and tables. Once a desired DNA sequence is inserted into the nuclear DNA of a cell, the location of an insertion region as well as the frequency with which the desired DNA sequence has been inserted into the nuclear genome can be identified by methods well known to those skilled in the art.

Desired DNA sequences can be inserted into nuclear DNA of a cell to enhance the resistance of a cloned transgenic animal to particular parasites, diseases, and infectious agents. Examples of parasites include worms, flies, ticks, and fleas. A transgene can confer resistance to a particular parasite or disease by completely abrogating or partially alleviating symptoms of the disease or parasitic condition or by producing a protein which controls the parasite or disease. Examples of infectious agents include bacteria, fungi, and viruses. Examples of diseases include Atrophic rhinitis, Cholera, Leptospirosis, Pseudorabies, Pasturellosis, and Brucellosis. These examples are not limiting and the invention relates to any disease or parasite or infectious agent known in the art. See, e.g., Hagan & Brunets Infectious Diseases of Domestic Animals (7th edition), Gillespie & Timoney, 1981, Cornell University Press, Ithaca N.Y.

A wide variety of transcriptional and translational regulatory sequences may be inserted into nuclear DNA of a nuclear donor cell. Transcriptional and translational regulatory signals may be derived from viral sources, such as adenovirus, bovine papilloma virus, cytomegalovirus, simian virus or the like, whereas the regulatory signals can be associated with a particular gene sequence having a potential for high levels of expression. Additionally, promoters from mammalian expression products, such as actin, casein alpha-lactalbumin, uroplakin, collagen, myosin, and the like, may be employed. Transcriptional regulatory signals may be selected which allow for repression or activation, so that expression of a gene product can be modulated. Of interest are regulatory signals which can be repressed or initiated by external factors such as chemicals or drugs. These examples are not limiting and the invention relates to any regulatory elements. Other examples of regulatory elements are described herein.

A variety of proteins and polypeptides can be encoded by a gene harbored within a DNA construct suitable for creating transgenic cells. Those proteins or polypeptides include hormones, growth factors, enzymes, clotting factors, apolipoproteins, receptors, drugs, pharmaceuticals, bioceuticals, nutraceuticals, oncogenes, tumor antigens, tumor suppressors, cytokines, viral antigens, parasitic antigens, bacterial antigens and chemically synthesized polymers and polymers biosynthesized and/or modified by chemical, cellular and/or enzymatic processes. Specific examples of these compounds include proinsulin, insulin, growth hormone, androgen receptors, insulin-like growth factor I, insulin-like growth factor II, insulin growth factor binding proteins, epidermal growth factor, TGF-α, TGF-β, dermal growth factor, platelet derived growth factor (PDGF), angiogenesis factors (e.g., acidic fibroblast growth factor, basic fibroblast growth factor, and angiogenim), angiogenesis inhibitors (e.g., endostatin and angiostatin), matrix proteins (Type IV collagen, Type VII collagen, laminin), oncogenes (ras, fos, myc, erb, src, sis, jun), E6 or E7 transforming sequence, p53 protein, cytokine receptor, IL-1, IL-6, IL-8, IL-2, α, β, or γ IFN, GMCSF, GCSF, viral capsid protein, and proteins from viral, bacterial and parasitic organisms. Other specific proteins or polypeptides which can be expressed include: phenylalanine hydroxylase, α-1-antitrypsin, cholesterol-7β-hydroxylase, truncated apolipoprotein B, lipoprotein lipase, apolipoprotein E, apolipoprotein Al, LDL receptor, scavenger receptor for oxidized lipoproteins, molecular variants of each, VEGF, and combinations thereof. Other examples are antibodies (monoclonal or polyclonal), antibody fragments, clotting factors, apolipoproteins, drugs, tumor antigens, viral antigens, parasitic antigens, monoclonal antibodies, and bacterial antigens. One skilled in the art readily appreciates that these proteins belong to a wide variety of classes of proteins, and that other proteins within these classes or outside of these classes can also be used. These are only examples and are not meant to be limiting in any way.

A pig prepared by a method in accordance with any aspect of the present invention may be used as a source of tissue for transplantation therapy. Similarly, a pig embryo prepared in this manner or a cell line developed therefrom may also be used in cell-transplantation therapy. Accordingly, there is provided in a further aspect of the invention a method of therapy comprising the administration of porcine cells to a patient, wherein the cells have been prepared from an embryo or animal prepared by a method as described above. This aspect of the invention extends to the use of such cells in medicine, e.g. cell-transplantation therapy, and also to the use of cells derived from such embryos in the preparation of a cell or tissue graft for transplantation. The cells may be organized into tissues, for example, heart, lung, liver, kidney, pancreas, corneas, nervous (e.g. brain, central nervous system, spinal cord), skin, or the cells may be blood cells (e.g. haemocytes, i.e. red blood cells, leucocytes) or haematopoietic stem cells or other stem cells (e.g. bone marrow). A method of the present invention will therefore also find utility in the preparation of xenografts. These methods might include in vitro differentiation of embryonic cells for therapeutic transplantation into a patient, including situations where the cells are genetically modified to correct a medical defect. Such applications might include treatment of diseases such as diabetes, Parkinson's disease, motor neurone disease, multiple sclerosis, AIDS etc, or disease conditions characterized by a loss of function in the cells or an organ of an affected individual.

Because of the human antibody induced hyperacute rejection of natural porcine tissues, various strategies are employed to modify the tissue to avoid transplant rejection. In one particular embodiment, the—1,3-galactosyltransferase porcine gene in pigs is knocked out (i.e. the expression of the gene is suppressed) to minimize the risk or incidence of hyperacute rejection. Knock out methods are known in the art, and are described in detail in PCT publication no. WO 98/57538 of Machaty, et al.

Stem Cell Applications

The recipient cell into which the donor nucleus has been transferred may be cultured in vitro or in vivo until a suitable stage in embryonic development is reached. The invention includes the derivation of a cell line from desired cells of the embryo, e.g. inner cell mass cells, for example in the derivation of a stem cell line. Suitably, the embryo may be cultured to the blastocyst stage.

The subject embryonic or stem-like cells may be used to obtain any desired differentiated cell type, by fusing donor cells with an enucleated oocyte, obtaining embryonic or stem-like cells as described above, and culturing such cells under conditions which favor differentiation to the desired cell type. Exemplary differentiated cells include hematopoietic stem cells and neural cells for the treatment of AIDS, leukemia, Parkinson's disease, Alzheimer's disease, ALS and cerebral palsy, among others.

Double Nuclear Transfer

An embryo resulting from a NT process can be manipulated in a variety of manners. The invention relates to cloned embryos, cells, cell lines, fetuses, and animals that arise from at least one nuclear transfer. Two or more NT procedures may be performed to enhance nuclear transfer efficiency of totipotent embryo, fetus, and animal production and/or placental development. Incorporating two or more NT cycles into methods for cloned embryos, fetuses, and animals can provide further advantages. For example, incorporating multiple NT procedures provides a method for multiplying the number of cloned embryos, fetuses, and animals. Moreover, gene targeting methods require that both copies of a given gene in a diploid cell be targeted in order to knock out or replace the gene. Such methods may require two or more NT procedures in order to efficiently target the gene. The skilled artisan will understand that the methods required for such manipulations will vary, depending on the species of interest.

For NT techniques that incorporate two or more NT cycles, one or more of the NT cycles may be preceded, followed, and/or carried out simultaneously with an activation step. As defined previously herein, an activation step may be accomplished by electrical and/or non-electrical means as defined herein. An activation step may also be carried out at the same time as a NT cycle (e.g., simultaneously with the NT cycle) and/or an activation step may be carried out prior to a NT cycle. Cloned embryos resulting from a NT cycle can be (1) disaggregated or (2) allowed to develop further.

Breeding Programs

The methods of the present invention are preferably performed in the context of an animal breeding operation, in which one or more of the animals produced by the present processes are introduced to a herd of animals for selective mating. Several characteristics can be used to define such an operation. For example, because the oocytes that are not preferentially selected for nuclear transfer are not of further commercial use, they are typically discarded. Thus, in one aspect the invention further provides discarding those oocytes that are not preferentially selected.

As another example, it will be appreciated that the use of the invention in a selective cloning and breeding program will result in the production of batches of clones at various fetal developmental stages. Thus, in another aspect the invention further provides nurturing a batch of one or more pregnant female recipients of nuclear transfer embryos, wherein all of such females within a batch are the recipients of embryos prepared from oocytes preferentially selected according to the processes of the instant invention. In still other embodiments, the breeding operation into which the instant process is integrated will comprise multiple batches of pregnant females in which all of such females within a batch are the recipients of embryos prepared from oocytes preferentially selected according to the processes of the instant invention. As used herein, the term “batch” refers to one or more animals into which have been implanted nuclear transfer embryos deriving from one batch of oocytes. A batch of oocytes can refer to a plurality of oocytes derived from a follicle, or to a plurality of oocytes derived from the follicles of one animal. Preferably, however, the operation is operated in batch-runs, and the term “batch” assumes its conventional meaning with reference to the distinction between a batch and continuous process.

### EXAMPLES

Materials and Methods

Donor Cells

Porcine fibroblasts were harvested from an ear skin biopsy obtained from an 8 months old pig. Miyoshi et al., Development of porcine embryos reconstituted with somatic cells and enucleated metaphase I and II oocytes matured in a protein free medium, BMC Dev Biol 2001, 1:12. Cells were cultured in DMEM/F-12 medium (Sigma Chemical Co., St. Louis, Mo.) supplemented with 20% (v:v) FCS (Biowhittaker Inc., Walkersville, Md.) under 5% CO₂ in air at 37° C. After reaching confluence, cells were passaged. Passage 2 fibroblasts were trypsinized, suspended in the culture medium with 10% (v:v) dimethyl sulfoxide (Sigma) and stored as frozen aliquots. Donor cells were used for nuclear transfer between passages 2 and 5 of culture. The cells were allowed to grow to confluency by culturing for 6 days and a single cell suspension was prepared by standard trypsinization immediately prior to nuclear transfer. The cells were used for nuclear transfer within 5 days after reaching confluence. Analysis of the cell cycle stages by using flow cytometry as explained in our earlier experiment (Miyoshi et al. (2001) (supra)) revealed that 91.2±0.2% of the cells were at the GO/G 1 phase.

In Vitro Maturation of Oocytes

Ovaries were collected from prepubertal gilts at a local slaughterhouse and transported to the laboratory at 30-35° C. Antral follicles (2-5 mm in diameter) were aspirated with an 18-gauge needle using vacuum suction (40 mm Hg). Aspirated oocytes that had an evenly granulated cytoplasm and were surrounded by at least three uniform layers of compact cumulus cells were selected and washed four times in Hepes-buffered TCM- 199 (Gibco BRL, Grand Island, N.Y.) supplemented with 0.4% (w:v) BSA (Sigma). Oocytes were cultured in 4-well plates (Nunc, Roskilde, Denmark) containing 500 μl of each medium. TCM-199 (Gibco) supplemented with 3.05 mM D-glucose, 0.91 mM Na-pyruvate, 0.57 mM cysteine, 10 ng/ml epidermal growth factor (Sigma), 10 IU/ml eCG (Sioux Biochemicals, Sioux Center, Iowa), 10 IU/ml hCG (Intervet, Millsboro, Del.), and 10% (v:v) porcine follicular fluid was used as a basic maturation medium.

Nuclear Transfer

At the end of maturation, cumulus cells were removed by vortexing with 0.1% (w:v) hyaluronidase (Sigma) and denuded oocytes were transferred into 100 μl of Hepes-buffered TCM-199 with the osmolarity adjusted to 300 mOsm by adding sorbitol supplemented with 7.5 μg/ml cytochalasin B (Sigma) and 10% FCS (manipulation medium). The first polar body and MII plate were removed by aspiration with a 15-μm inner diameter glass pipette. The oocytes had been previously stained in the manipulation medium supplemented with 5 μg/ml Hoechst 33342 (Sigma) for 20 min and confirmation of successful enucleation was achieved by visualizing the cytoplast and removed cytoplasm under ultraviolet light. After enucleation, a donor cell was inserted into the perivitelline space of each enucleated oocyte using the same glass pipette. Cell-oocyte complexes were washed in TCM-199 supplemented with 10% FCS, transferred to the same medium and kept in a CO₂ incubator adjusted to 5% CO₂ in air at 38.5° C. until fusion.

The chamber for fusion was a 100 mm dish filled with 15 ml of Zimmerman fusion medium, Zimmerman, et al., Electric field-induced cell-to-cell fusion. J Membr Biol 1982, 67: 165-182. Two stainless steel wires (100 μm diameter) were used as electrodes and they were attached to micromanipulators. The single cell-oocyte complex was sandwiched between the electrodes and oriented with the contact surface between the cytoplast and the donor cell perpendicular to the electrodes. The distance between the electrodes was about 100 μm. Membrane fusion was induced by applying a single direct current pulse of 250 V/mm for a duration of 20 μsec with a prepulse of alternating current field of 5 V, 1 MHz for 2 sec using an LF 101 Fusion Machine (TR Tech Co., Tokyo, Japan). Following the fusion pulse, the complexes were washed in G1.2 medium, Gandhi, et al., Substrate utilization in porcine embryos cultured in NCSU23 and G1.2/G2.2 sequential culture media, Mol Reprod Dev 2001, 58: 269-275 and cultured for a period of 1 h in 100 μl of the same medium. Fusion was then determined by microscopic examination.

Activation and Culture of Reconstructed Embryos

At 3 h after fusion, fused embryos were washed once in Zimmerman fusion medium and then placed between 2 wire electrodes (1 mm apart) of the fusion chamber slide with 15 ml of the medium. Direct current pulses of 75 V/mm were applied twice to the embryos for a duration of 60 μsec at intervals of 5 sec. Activated embryos were incubated in 100 μl of G1.2 medium supplemented with 7.5 μg/ml cytochalasin B for 2 h after activation to prevent extrusion of polar bodies, Miyoshi, et al. (2000) (supra), and then transferred into G1.2 medium. After culture in G 1.2 medium for 3 days, the embryos were transferred into 100 μl of G2.2 medium, Gandhi, et al., (2001) (supra), and continued to be cultured. The embryos were examined for cleavage and blastocyst formation at 2 and 7 days of culture, respectively. At the end of the culture period, all blastocysts were counted for nuclei after Hoechst staining. The blastocysts were placed on slides with a drop of mounting medium consisting of glycerol and PBS (9: 1) containing 0.1 mg/ml Hoechst 33342. A cover slip was placed on the top of the blastocysts and the edge was sealed with fingernail polish. The numbers of nuclei were counted under ultraviolet light.

Experimental Studies

In order to determine the optimal treatment period for dbcAMP induced GV stage synchronization, oocytes were cultured in the basic maturation medium with or without 1 mM dbcAMP for 16, 20, 24, and 42 h in Experiment 1. After culture for each period, degenerated oocytes with lysed cytoplasmic membranes were removed. The remainder were mounted, fixed for 72 h in 25% (v:v) acetic acid in ethanol at room temperature, stained with 1% (w:v) orcein in 45% (v:v) acetic acid, and examined for meiotic progression under a phase-contrast microscope.

In experiment 2, oocytes were cultured in the basic maturation medium with or without 1 mM dbcAMP for 20 h and then transferred into the medium without dbcAMP and hormonal supplements. Oocytes continued to be cultured for additional 4, 10, 16, and 22 h and examined for meiotic progression to the M II stage by staining with 5 μg/ml Hoechst 33342.

In Experiment 3, oocytes were cultured in the basic maturation medium for 20 h and then transferred into the medium without hormonal supplements. M II oocytes collected at 24, 30, and 42 h post initiation of maturation were used in the nuclear transfer procedure and the resulting embryos were cultured in vitro and developmental rates among groups were compared.

In Experiment 4, oocytes were cultured under the same conditions as those in Experiment 3 and M II oocytes were collected at 24 and 42 h of maturation. They were cultured in TCM-199 supplemented with 10% FCS for 6-7 h and activated, which was the same timing as reconstruction of embryos in Experiment 3. Activated oocytes were cultured in vitro to examine developmental rates. The activation and culture methods were the same as those described above.

In Experiment 5, oocytes were cultured in the basic maturation medium with or without 1 mM dbcAMP for 20 h and then transferred into the medium without dbcAMP and hormonal supplements. Embryos reconstituted with M II oocytes collected at 42 h of maturation were cultured in vitro and their developmental abilities were compared.

Statistical Analysis

All percentage data were subjected to an arcsin transformation in each replicate and the transformed values were analyzed using one-way (Experiments 3, 4, and 5) or two-way (Experiments 1 and 2) ANOVA followed by Student-Newman-Keuls test. The numbers of cells in blastocysts were analyzed using Student t-test. A probability of P<0.05 was considered statistically significant.

Results

Experiment 1. Effect of Dibutyryl Cyclic AMP on Meiotic Resumption of Porcine Oocytes Matured In Vitro

There were no significant differences in percentages of degenerated oocytes in any treatment groups in this study. The addition of dbcAMP in the maturation medium increased (P<0.05) the percentage of GV oocytes at each culture period (Table 1). In the absence of dbcAMP, 42.6% of oocytes were at the GV stage at 16 h of culture. This percentage was significantly (P<0.05) higher than those at 24 h (9.3%) and 42 h (3.7%) of culture. In the presence of dbcAMP, the percentages of GV oocytes at 16 and 20 h of culture (77.8 and 72.9%, respectively) were significantly (P<0.05) higher than at 42 h (44.3%) of culture.

TABLE 1
Maturation of porcine oocytes cultured in a medium supplemented
with or without 1 mM dibutyryl cyclic AMP (dbcAMP).^{a}

| | Period | | | | |
|---|---|---|---|---|---|
| | of | No. of | No. of oocytes | No. of | No. of oocytes at |
| dbc- | culture | oocytes | degenerated | oocytes | the germinal |
| AMP | (h) | cultured | (%)^{b} | examined | vesicle stage (%)^{c} |
| − | 16 | 83 | 15 (18.1 ± 5.5) | 68 | 29 (42.6 ± 4.0)^{d} |
| | 20 | 84 | 17 (20.2 ± 3.4) | 60 | 9 (15.0 ± 10.2)^{de} |
| | 24 | 82 | 18 (22.0 ± 0.9) | 54 | 5 (9.3 ± 6.1)e |
| | 42 | 84 | 20 (23.8 ± 7.9) | 54 | 2 (3.7 ± 4.2)^{e} |
| + | 16 | 79 | 15 (19.0 ± 1.6) | 63 | 49 (77.8 ± 3.2)^{d} |
| | 20 | 80 | 10 (12.5 ± 7.3) | 59 | 43 (72.9 ± 10.4) |
| | 24 | 82 | 19 (23.2 ± 4.5) | 61 | 33 (54.1 ± 7.0)^{de} |
| | 42 | 80 | 16 (20.0 ± 7.6) | 61 | 27 (44.3 ± 2.6)^{e} |

^{a}Experiments were repeated 3 times.
^{b}Percentage (mean ± SEM) of oncytes cultured.
^{c}Percentage (mean ± SEM) of oocytes examined. Values are significantly different between presence and absence of dbcAMP at each culture period (P < 0.05).
^{d,e}Values with different superscripts within each treatment (presence or absence of dbcAMP) are significantly different (P < 0.05).

Experiment 2. In Vitro Maturation of Porcine Oocytes Treated with or without Dibutyryl Cyclic AMP

The addition of dbcAMP in the maturation medium decreased (P<0.05) the percentage of M II oocytes at 10 h of additional culture (Table 2). In the absence of dbcAMP, 36.3% of oocytes reached the M II stage at 4 h of additional culture. This percentage was significantly (P<0.05) lower than those at 10 h (71.4%), 16 h (80.0%), and 22 h (78.0%) of additional culture. In the presence of dbcAMP, the percentages of M II oocytes at 4 and 10 h of additional culture (35.3 and 29.8%, respectively) were significantly (P<0.05) lower than that at 16 h (67.5%) of additional culture.

TABLE 2
Maturation of porcine oocytes cultured in a medium supplemented
with or without 1 mM dibutyryl cyclic AMP (dbcAMP) for 20 h and then
continued to be cultured in the medium without dbcAMP.^{a}

| | Period | | | | |
|---|---|---|---|---|---|
| | of | | | | |
| | ad- | | | No. of | |
| | itional | No. of | No. of oocytes | oocytes | No. of oocytes at |
| dcb- | culture | oocytes | degenerated | ex- | the metaphase II |
| AMP | (h) | cultured | (%)^{b} | amined | stage (%)^{c} |
| − | 4 | 104 | 13 (12.5 ± 2.2) | 91 | 33 (36.3 ± 8.7)^{d} |
| | 10 | 99 | 15 (15.2 ± 1.8) | 84 | 60 (71.4 ± 8.9)^{e} |
| | 16 | 104 | 19 (18.3 ± 5.5) | 85 | 68 (80.0 ± 5.0)^{e} |
| | 22 | 104 | 22 (21.2 ± 7.2) | 82 | 64 (78.0 ± 10.4)^{e} |
| + | 4 | 98 | 13 (13.3 ± 2.4) | 85 | 30 (35.3 ± 9.2)^{d} |
| | 10 | 103 | 19 (18.4 ± 4.0) | 84 | 25 (29.8 ± 1.4)^{d} |
| | 16 | 105 | 25 (23.8 ± 10.5) | 80 | 54 (67.5 ± 8.4)^{e} |
| | 22 | 106 | 25 (23.6 ± 6.4) | 81 | 69 (85.2 ± 5.0)^{e} |

^{a}Experiments were repeated 3 times.
^{b}Percentage (mean ± SEM) of oocytes cultured.
^{c}Percentage (mean ± SEM) of oocytes examined. Values are significantly different between presence and absence of dbcAMP at 10 h of additional culture (P < 0.05).
^{d,e}Values with different superscripts within each treatment (presence or absence of dbcAMP) are significantly different (P < 0.05).

Experiment 3. In Vitro Development of Porcine Embryos Reconstituted with Metaphase II Oocytes Collected at Various Times post Initiation of Maturation

There were no significant differences in percentages of fused cell-oocyte complexes (86.8-93.9%) and cleaved embryos (29.8-42.9%) among different maturation periods (Table 3). However, when M II oocytes recovered at 24 h of maturation were used as recipients, the percentage (14.1%) of cloned embryos developing to the blastocyst stage was significantly (P<0.05) higher than those of embryos reconstituted with 30-h-matured (3.0%) and 42-h-matured (6.0%) oocytes. There were no significant differences in mean numbers (28.8-58.8 cells) of cells in the blastocysts among different maturation periods.

TABLE 3
Effect of maturation periods of recipient oocytes on development of
porcine embryos reconstituted with somatic cells.^{a}

| Period of | No. of | No. of | No. (%)^{c} of embryos developed to | | Mean no. ± SEM |
|---|---|---|---|---|---|
| matura- | complexes | complexes | ≧2-cell | Blastocyst | of cells in |
| tion (h) | treated | fused (%)^{b} | (2)^{d} | (7)^{d} | blastocysts |
| 24 | 173 | 156 (90.2 ± 3.8) | 64 (41.0 ± 1.0) | 22 (14.1 ± 2.3)^{e} | 49.5 ± 6.4 |
| 30 | 179 | 168 (93.9 ± 2.3) | 50 (29.8 ± 4.3) | 5 (3.0 ± 0.8)^{f} | 28.8 ± 3.9 |
| 42 | 250 | 217 (86.8 ± 7.0) | 93 (42.9 ± 5.8) | 13 (6.0 ± 1.0)^{f} | 58.8 ± 9.8 |

^{a}Experiments were repeated 3 times.
^{b}Percentage (mean ± SEM) of complexes treated.
^{c}Percentage (mean ± SEM) of complexes fused.
^{d}Numbers in parentheses indicate the time of examination (days of culture).
^{e,f}Values with different superscripts are significantly different (P < 0.05).

Experiment 4. Effect of Maturation Periods on In Vitro Development ofporcine Oocytes after Activation

There were no significant differences in percentages of cleaved oocytes (32.9-45.6%) and oocytes developed to blastocysts (6.8-7.6%) after activation and in mean numbers (42.4-54.0 cells) of cells in the blastocysts between different periods of maturation (Table 4).

TABLE 4
Effect of maturation periods on development of porcine oocytes after
activation.^{a}

| | | No. (%)^{b} of | | |
|---|---|---|---|---|
| Period of | No. of | oocytes developed to | | Mean no. ± SEM |
| maturation | oocytes | ≧2-cell | Blastocyst | of cells in |
| (h) | cultured | (2)^{c} | (7)^{c} | blastocysts |
| 24 | 73 | 24 (32.9 ± 10.6) | 5 (6.8 ± 3.8) | 42.4 ± 12.7 |
| 42 | 79 | 36 (45.6 ± 4.3) | 6 (7.6 ± 2.2) | 54.0 ± 13.1 |

^{a}Experiments were repeated 3 times.
^{b}Percentage (mean ± SEM) of oocytes cultured.
^{c}Numbers in parentheses indicate the time of examination (days of culture).

Experiment 5. In Vitro Development of Porcine Embryos Reconstituted with Metaphase II Oocytes Treated with or without Dibutyryl Cyclic AMP

There were no significant differences in percentages of fused cell-oocyte complexes (78.8-80.0%), cleaved embryos (33.3-41.4%) and embryos developed to blastocysts (1.7-5.7%) between presence and absence of dbcAMP treatment (Table 5). In addition, there was no significant difference in mean numbers (49.0-77.7 cells) of cells in the blastocysts between presence and absence of dbcAMP treatment.

TABLE 5
Development of porcine embryos reconstituted with 42-h-matured
oocytes treated with or without dibutyryl cyclic AMP.^{a}

| | | | No. (%)^{c} | | |
|---|---|---|---|---|---|
| Dibutyryl | No. of | No. of | of embryos developed to | | Mean no. ± SEM |
| cyclic | complexes | complexes | ≧2-cell | Blastocyst | of cells in |
| AMP | treated | fused (%)^{b} | (2)^{d} | (7)^{d} | blastocysts |
| — | 156 | 123 (78.8 ± 1.0) | 41 (33.3 ± 9.2) | 7 (5.7 ± 2.4) | 77.7 ± 16.2 |
| + | 145 | 116 (80.0 ± 4.5) | 48 (41.4 ± 8.3) | 2 (1.7 ± 1.5) | 49.0 ± 7.0 |

^{a}Experiments were repeated 3 times.
^{b}Percentage (mean ± SEM) of complexes treated.
^{c}Percentage (mean ± SEM) of complexes fused.
^{d}Numbers in parentheses indicate the time of examination (days of culture).

Throughout this application, various publications are referenced. The disclosures of these publications in their entireties are hereby incorporated by reference into this application in order to more fully describe the state of the art to which this invention pertains.

Throughout this specification the word ‘comprise,’ or variations such as ‘comprises’ or ‘comprising,’ will be understood to imply the inclusion of a stated element, integer or step, or group of elements, integers or steps, but not the exclusion of any other element, integer or step, or group of elements, integers or steps.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1) A method of producing a cloned or genetically modified non-human mammalian embryo comprising:
a) providing a cell culture comprising a plurality of in vitro matured oocytes;
b) preferentially selecting from the cell culture a rapidly matured oocyte;
c) transferring DNA from a donor cell derived from non-human mammalian tissue to the matured oocyte to form an embryo.

2) A method of producing a cloned or genetically modified non-human mammalian embryo comprising:
a) providing a cell culture comprising one or more oocytes that have been matured in vitro for a rapid maturation period;
b) selecting a matured oocyte from the cell culture; and
c) transferring DNA from one or more donor cells derived from non- human mammalian tissue to the matured oocyte to form an embryo.

3) A method of producing a cloned or genetically modified non-human mammalian embryo comprising:
a) providing a cell culture comprising a plurality of vitro matured oocytes;
b) preferentially selecting a matured oocyte from the culture that attained developmental competence in vivo;
c) transferring DNA from a donor cell derived from non-human mammalian tissue to the matured oocyte to form an embryo.

4) A method of producing a cloned or genetically modified non-human mammalian embryo comprising:
a) providing a cell culture comprising one or more oocytes that have been matured in vitro for a period of time approximating or less than the time required of a developmentally competent to reach maturation in vitro;
b) selecting a matured oocyte from the cell culture; and
c) transferring DNA from a donor cell derived from non-human mammalian tissue to the matured oocyte to form an embryo.

5) A method of producing a cloned or genetically modified non-human mammalian embryo comprising:
a) providing a cell culture comprising one or more oocytes and a cell cycle inhibitor;
b) selecting a matured oocyte from the cell culture; and
c) transferring DNA from a non-human donor cell to the matured oocyte to form an embryo.

6) The method of claim 1, further comprising activating the nuclear transfer unit.

7) The method of claim 1, further comprising activating the nuclear transfer embryo by contacting the nuclear transfer unit with ionomycin, cytochalasin B, and cycloheximide.

8) The method of claim 1 further comprising transferring said embryo into a recipient female so as to produce a fetus that undergoes full fetal development and parturition to generate a live-born animal.

9) The method of claim 1 further comprising:
a) preferentially selecting from the cell culture one or more additional rapidly matured oocytes;
b) transferring DNA from one or more additional donor cells derived from non-human mammalian tissue to the one or more additional rapidly matured oocytes to form one or more additional embryos.

10) The method of claim 9 wherein at least 8% (P<0.05) of the embryos are transferred to a recipient.

11) The method of claim 9 wherein at least 10% (P<0.05) of the nuclear transfer embryos are transferred to a recipient.

12) The method of claim 9 wherein at least 12% (P<0.05) of the nuclear transfer embryos are transferred to a recipient.

13) The method of claim 9 further comprising harvesting said oocytes from a porcine follicle larger than about 2 mm in diameter.

14) The method of claim 9 further comprising harvesting said oocytes from a porcine follicle, wherein said oocyte is greater than about 110 μm in diameter when harvested from said follicle.

15) The method of claim 1 wherein the mammalian embryo is porcine or bovine.

16) The method of claim 1 wherein the rapidly matured oocyte reached maturity faster than about 40% of the oocytes in the cell culture.

17) The method of claim 1 wherein the rapidly matured oocyte reached maturity faster than about 60% of the oocytes in culture.

18) The method of claim 1 wherein the rapidly matured oocyte reached maturity faster than about 80% of the oocytes in culture.

19) The method of claim 1 wherein the mammal is porcine, and the oocyte matured in from about 6 to about 36 hours.

20) The method of claim 1 wherein the mammal is porcine, and the oocyte matured in from about 10 to about 32 hours.

21) The method of claim 1, wherein the mammal is porcine, and the oocyte matured in vivo in from about 20 hours to about 28 hours.

22) The method of claim 1, wherein the mammal is bovine, and the oocyte matured in vivo in from about 6 hours to about 40 hours.

23) The method of claim 1, wherein the mammal is bovine, and the oocyte matured in vivo in from about 8 hours to about 28 hours.

24) The method of claim 1, wherein the mammal is bovine, and the oocyte matured in vivo in from about 10 hours to about 16 hours.

25) The method of claim 1 wherein the oocyte reached maturation in less than the post-GV-II maturation period.

26) The method of claim 1 wherein the oocyte reached maturation in a time equal to or less than the post-GV-III maturation period.

27) The method of claim 5 wherein the cell cycle inhibitor is dbcAMP.

28) The method of claim 1 wherein said DNA from said donor cell is transgenically modified.

29) The method of claim 1 wherein said embryo is cultured under conditions that result in development into a blastocyst or post-blastocyst stage embryo, further comprising isolating totipotent cells from said blastocyst or post-blastocyst embryo and expanding said cells in culture to produce embryonic stem cells.
